(19) United States
(12) Patent Application Publication
(10) Pub. No.: US 2023/0338336 A1
(43) Pub. Date: Oct. 26, 2023

(54) METHODS OF TREATING CONDITIONS RELATED TO THE S1P1 RECEPTOR

(71) Applicant: Arena Pharmaceuticals, Inc., San Diego, CA (US)

(72) Inventors: Catherine M Crosby, San Diego, CA (US); Gurpreet Ahluwalia, Santa Ana, CA (US); Andrew Christopher Wesley Selfridge, Buckinghamshire (GB)

(21) Appl. No.: 17/790,632

(22) PCT Filed: Jan. 6, 2021
(86) PCT No.: PCT/US2021/012367

§ 371 Date: Jul. 1, 2022

Related U.S. Application Data
(60) Provisional application No. 62/957,535, filed on Jan. 6, 2020.

(51) Int. Cl. A61K 31/403; A61K 9/48; A61K 9/20; A61P 17/14

(52) U.S. Cl. CPC A61K 31/403; A61K 9/4825; A61K 9/2054; A61K 9/2018; A61K 9/2013; A61P 17/14; A61K 9/2059

(57) ABSTRACT

Provided are methods of treatment of alopecia areata comprising prescribing and/or administering to an individual in need thereof a standard dose of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof.

Provided are methods useful in the treatment of alopecia areata.

Alopecia areata is the most common cause of inflammation-induced hair loss. In the United States, alopecia areata affects approximately 4.5 million people and the incidence rate is estimated to 0.1%-0.2%. The clinical presentation of hair loss is variable. The most common presentation is alopecia areata (90%) with one or more bald circumscribed patches on the scalp. Typically, the affected skin has no sign of inflammation. Short fragile hairs (so-called exclamation point) are often seen in the periphery of lesions. Approximately 7% of patients may progress to alopecia totalis with a total loss of scalp hair or alopecia universalis with hair loss on the scalp and body. A few patients experience a diffuse type of alopecia areata or preferential loss of pigmented hair. Nail changes, including trachonychia, pitted nail, or longitudinal ridges, are seen in 20% of patients. Spontaneous regrowth of hair may occur in 50% of patients within one year and particularly in mild cases.

Alopecia areata has an unpredictable course and there is currently no approved therapy specifically for the disease. Current evidence-based therapy is limited to topical (including injection) or systemic corticosteroids and sensitizing agents such as diphenylcyclopropenone and dinitrochlorobenzene. However, this treatment is only feasible or small areas, and long-term use is often unacceptable and with disappointing efficacy. There is no evidence of efficacy for systemic immunosuppressants such as methotrexate, mycophenolate mofitile, cyclosporine A, or azathioprine. Nor is there robust evidence for topical tacrolimus, cryotherapy, or ultraviolet light A combined with oral psoralens (PUVA).

Alopecia areata has a significant negative impact on quality of life and self-esteem and there is a large unmet medical need for new effective treatment options, as current therapies often provide only transient or marginal symptomatic relief. The present disclosure satisfies this need and provides related advantages as well.

Citation of any reference throughout this application is not to be construed as an admission that such reference is prior art to the present application.

### SUMMARY

Provided is a method of treating alopecia areata in an individual in need thereof comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof. In some embodiments, the individual has severe alopecia areata. In some embodiments, the individual has moderate alopecia areata. In some embodiments, the individual is administered a therapeutically effective amount of Compound 1, or a pharmaceutically acceptable salt thereof, that is equivalent to about 0.5 to about 5.0 mg of Compound 1. In some embodiments, the individual is administered a therapeutically effective amount of Compound 1, or a pharmaceutically acceptable salt thereof, that is equivalent to 2 mg of Compound 1. In other embodiments, the individual is administered a therapeutically effective amount of Compound 1, or a pharmaceutically acceptable salt thereof, that is equivalent to 3 mg of Compound 1. In some embodiments, the Compound 1, or a pharmaceutically acceptable salt thereof, is administered orally. According to some embodiments, the therapeutically effective amount of Compound 1, or a pharmaceutically acceptable salt thereof, is administered at a frequency of once per day. According to some embodiments, the administering results in no serious adverse events. In some embodiments, the Compound 1, or a pharmaceutically acceptable salt thereof, is administered without substantially inducing an acute heart rate reduction or heart block in the individual. In some embodiments, the individual has greater than or equal to 50% scalp hair loss. In some embodiments, the method is therapeutically effective to achieve at least a 50% improvement from the individual's baseline Severity of Alopecia Tool (SALT) score. In some embodiments, the method is therapeutically effective to achieve at least a 50% improvement from the individual's baseline SALT score in a period of time of at least about 24 weeks.

Also provided is a method of inducing hair regrowth in an individual diagnosed with alopecia areata comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof. In some embodiments, the individual has severe alopecia areata. In some embodiments, the individual has moderate alopecia areata. In some embodiments, the individual is administered a therapeutically effective amount of Compound 1, or a pharmaceutically acceptable salt thereof, that is equivalent to about 0.5 to about 5.0 mg of Compound 1. In some embodiments, the individual is administered a therapeutically effective amount of Compound 1, or a pharmaceutically acceptable salt thereof, that is equivalent to 2 mg of Compound 1. In other embodiments, the individual is administered a therapeutically effective amount of Compound 1, or a pharmaceutically acceptable salt thereof, that is equivalent to 3 mg of Compound 1. In some embodiments, the Compound 1, or a pharmaceutically acceptable salt thereof, is administered orally. According to some embodiments, the therapeutically effective amount of Compound 1, or a pharmaceutically acceptable salt thereof, is administered at a frequency of once per day. According to some embodiments, the administering results in no serious adverse events.

These and other aspects of the invention disclosed herein will be set forth in greater detail as the patent disclosure proceeds.

### BRIEF DESCRIPTION OF DRAWINGS

FIG. 1: Normal hair cycle is shown in panel A. The hair cycle in alopecia areata is shown in panel B. See Gilhar et al. NEJM 2012; 366: 1515-25.

FIG. 2: Examples of severity of alopecia areata: (a)>50% hair loss, (b)<50% hair loss, and (c) subject with alopecia totalis.

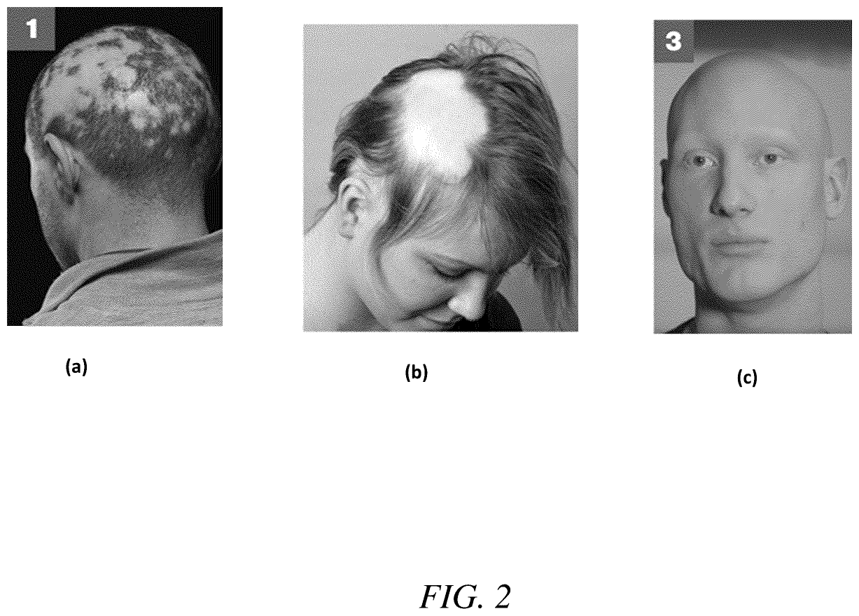

FIG. 3: The SALT I aid for determining scalp surface area is shown.

FIG. 4: The SALT II aid for determining scalp surface area is shown.

FIG. 5: Comparative images of hair follicle epithelium and S1P expression data in an AA patient sample versus a healthy patient sample are illustrated according to Example 3.

FIG. 6: Images of S1P₁⁺ CD8⁺ cells increase in AA patients are illustrated and shown through data according to Example 3.

FIG. 7: Illustrates the effect of Compound 1 on hair cycle staging and hair cycle score as compared to vehicle according to Example 3.

FIG. 8a: Demonstrates the effect of Compound 1 on MHC class I in the dermal cup, germinative hair matrix, and outer root sheath in anagen and catagen hair follicles as described in Example 3.

FIG. 8b: Demonstrates the effect of Compound 1 on MHC class I in the dermal cup, germinative hair matrix, and outer root sheath in anagen hair follicles as described in Example 3.

FIG. 9a: Shows the effect of Compound 1 on MICA in the dermal cup, germinative hair matrix, and outer root sheath in anagen and catagen hair follicles as described in Example 3.

FIG. 9b: Demonstrates the effect of Compound 1 on MICA in the dermal cup, germinative hair matrix, and outer root sheath in anagen hair follicles as described in Example 3.

FIG. 10: Illustrates the effect of Compound 1 on hair cycle staging and hair cycle score according to Example 3.

FIG. 11: Shows the effect of Compound 1 on MHC I in the dermal cup as described in Example 3.

FIG. 12: Shows the effect of Compound 1 on Perifollicular CD8+ and CD8+S1PR1+ cells per hair follicle as described in Example 3.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy)-1,2,3,4-tetrahydrocyclopenta[b]indol-3-yl)acetic acid including crystalline forms thereof.

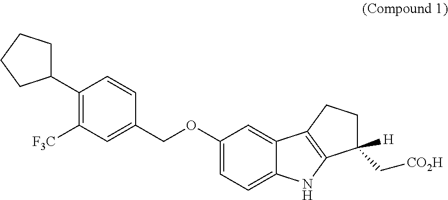 [SMILES: O=C(O)C[C@H]1CCc2c1[nH]c1ccc(OCc3ccc(C4CCCC4)c(C(F)(F)F)c3)cc21]

See PCT patent application, Serial No. PCT/US2009/004265, hereby incorporated by reference in its entirety. As a non-limiting example, Compound 1 may be present as an anhydrous, non-solvated crystalline form as described in WO 2010/011316 (incorporated by reference herein in its entirety). As another non-limiting example, an L-arginine salt of Compound 1 may be present as an anhydrous, non-solvated crystalline form as described in WO 2010/011316 and WO 2011/094008 (each of which is incorporated by reference herein in its entirety). As another non-limiting example, a calcium salt of Compound 1 may be present as a crystalline form as described in WO 2010/011316 (incorporated by reference herein in its entirety). Compound 1 is referred to in literature as etrasimod or APD334.

Compound 1, or a pharmaceutically acceptable salt, solvate, or hydrate thereof, is an orally administered, selective, synthetic sphingosine 1-phosphate (S1P) receptor 1, 4, 5 modulator. To date, Compound 1, or a pharmaceutically acceptable salt, solvate, or hydrate thereof, has been found to be safe and well-tolerated in approximately 281 adult subjects treated at various doses. Its safety and tolerability have been evaluated in Phase 1 studies with healthy adult subjects at single doses up to 5 mg and repeated doses up to 4 mg once daily (QD). In a Phase 2 dose-ranging study in UC patients, treatment with 2 mg QD for 12 weeks led to clinically meaningful and statistically significant endoscopic and symptomatic improvements versus placebo. Sustained beneficial effects of were observed for up to 46 weeks in the subsequent open-label extension study.

ADMINISTERING: As used herein, “administering” means to provide a compound or other therapy, remedy, or treatment such that an individual internalizes a compound.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number, or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or one or more condition symptoms, preventing one or more additional symptoms, ameliorating the underlying metabolic causes of one or more symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound 1 or a pharmaceutically acceptable salt, solvate, or hydrate thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is an abnormal liver function test, such as an elevated ALT & AST>2X ULN. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g., physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit, or ameliorate the disease, condition, or disorder.

INDIVIDUAL: As used herein, “individual” means any human. In some embodiments, a human individual is referred to a “subject” or “patient.”

ACUTE HEART RATE REDUCTION: As used herein, “acute heart rate reduction” means a heart rate decrease from normal sinus rhythm of, for example, 10 or more beats per minute (bpm), such as less than about 5 bpm, e.g., less than about 4 bpm or less than about 3 bpm or less than 2 bpm, that is maximal within a few hours, for example 1-3 hours, after drug administration, and thereafter the heart rate returns towards the pre-dose value.

NORMAL SINUS RHYTHM: As used herein, “normal sinus rhythm” means the sinus rhythm of the individual when not undergoing treatment. The evaluation of normal sinus rhythm is within the ability of a physician. A normal sinus rhythm will generally give rise to a heart rate in the range from 60-100 bpm.

DOSE: As used herein, “dose” means a quantity of Compound 1, or a pharmaceutically acceptable salt, solvate, or hydrate thereof, given to the individual for treating or preventing the disease or disorder at one specific time.

STANDARD DOSE: As used herein, “standard dose” means the dose of Compound 1, or a pharmaceutically acceptable salt, solvate, or hydrate thereof, that is given to the individual for treating or preventing the disease or disorder. The target dose may vary depending on the nature and severity of the disease to be treated.

THERAPEUTICALLY EFFECTIVE AMOUNT: As used herein, “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

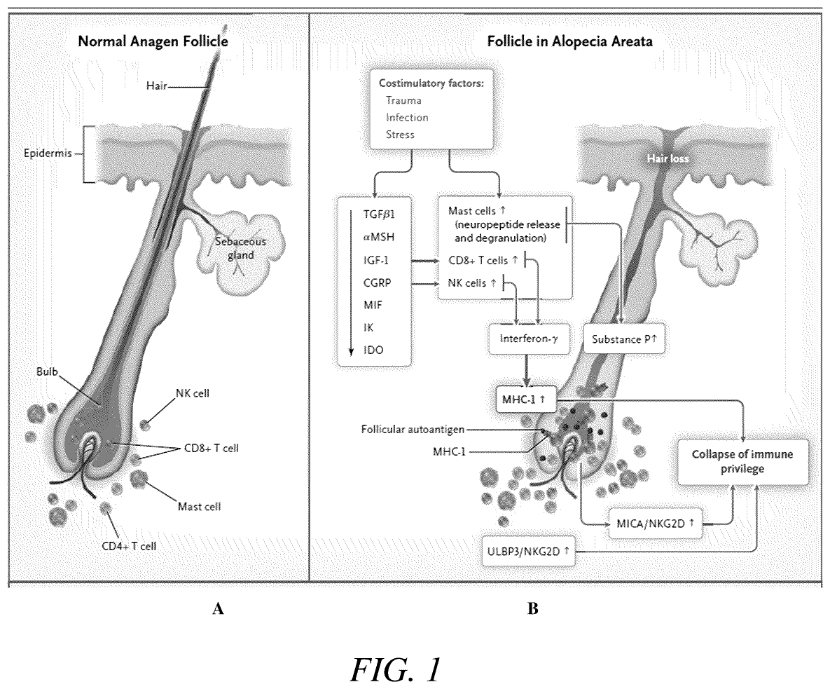

ALOPECIA AREATA: As used herein, “alopecia areata” or “AA” means a chronic T-cell mediated autoimmune skin disease leading to hair loss. The pathogenesis of alopecia areata is shown in FIG. 1. Hair may be lost more diffusely over the whole scalp; in which case the condition is called diffuse alopecia areata. Alopecia areata monolocularis describes baldness in only one spot. It may occur anywhere on the head. Alopecia areata multilocularis refers to multiple areas of hair loss. Ophiasis refers to hair loss in the shape of a wave at the circumference of the head. The disease may be limited only to the beard, in which case it is called alopecia areata barbae. If the person loses all the hair on the scalp, the disease is then called alopecia areata totalis. If all body hair is lost, the diagnosis then becomes alopecia areata universalis. Severe alopecia areata refers to 50% or more involvement of the entire scalp, alopecia totalis, and alopecia universalis. The present invention includes methods to treat all forms of alopecia areata.

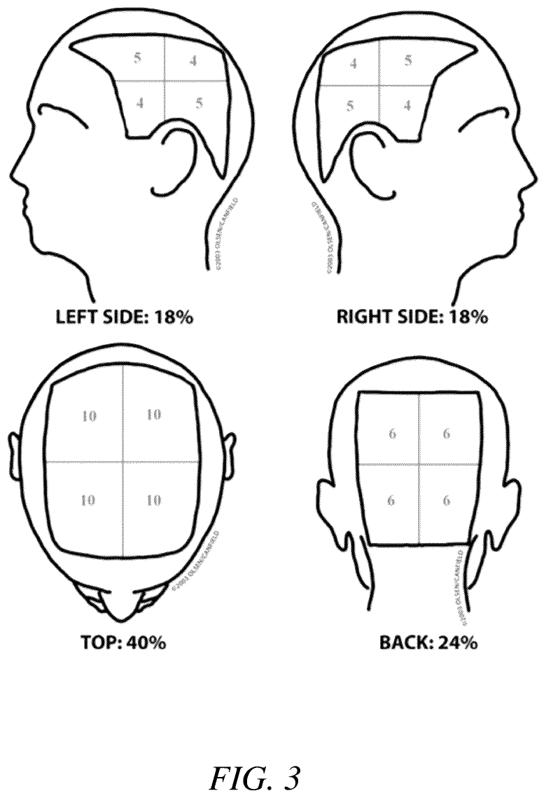

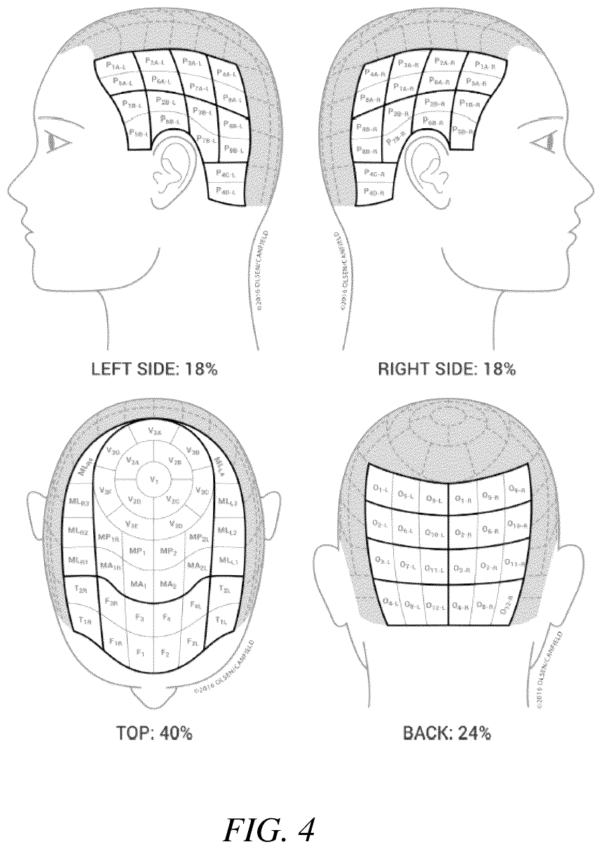

SALT SCORE: As used herein, “SALT score” refers to the Severity of Alopecia Tool. The SALT I score is a validated and widely used tool for determining degree of hair loss based on the percentage of scalp surface area involved on the top, back, and each side of the scalp (FIG. 3). Using the diagram in FIG. 3, an investigator or medical professional can determine the percent scalp hair loss in a given quadrant, multiply this by the total scalp area delineated by that quadrant, and sum the resultant numbers for each quadrant to give the total percent scalp hair loss with a maximum score of 100. The SALT II score is an updated tool which includes smaller increments of scalp coverage to facilitate the assessment of hair loss where small patches of hair loss predominate (FIG. 4). See, e.g., Olsen E A, Hordinsky M K, Price V H, et al. Alopecia areata investigational assessment guidelines—Part II. J Am Acad Dermatol. 2004; 51(3): 440-447; Olsen et al. (2016) J. Am. Acad. Dermatol. Research Letters 1268-1270; Olsen (2001) J. Am. Acad. Dermatol. 45(3 Suppl): S70-S80; and Olsen et al. (2003) Hair Science and Technology 2003: 251-254, each of which is incorporated by reference for all purposes. The SALT score, using either the original SALT I or SALT II, is determined by adding the percentage hair loss in the various areas of the scalp, for a maximum score of 100.

ALOPECIA AREATA SYMPTOM IMPACT SCALE (AASIS): As used herein “AASIS” can refer to a 13-item, disease-specific measure that asks AA patients about symptoms related to AA and how these symptoms interfere with daily functioning. Patients can be asked to rate how severe each of the following 7 symptoms pertaining to AA symptoms have been in the past week using an 11-point scale ranging from 0 “not present” to 10 “as bad as you can imagine”: 1) scalp hair loss, 2) body or eye lashes hair loss, 3) tingling/numbness of the scalp, 4) itchy or painful skin, 5) irritated skin, 6) feeling anxious or worry, or 7) feeling sad. Patients can also be asked to rate how severe each of the following 6 areas of daily functioning are interfered with by AA in the past week using an 11-point scale ranging from 0=“did not interfere” to 10 “interfered completely”: work, enjoyment of life, interaction with others, daily activities, sexual relationships, and quality of life. The overall scoring system ranges from of 0 to 130 with high scores indicative of greater AA symptom impact.

ALOPECIA AREATA-RELATED QUALITY OF LIFE INDEX (AA-QLI): As used herein the Alopecia Areata-Related Quality of Life Index (AA-QLI) refers to a disease-specific questionnaire developed to evaluate the impact of AA on quality of life. The results are presented on a scale varying between 0 and 84; a score of 0 represents the best quality of life, while a score of 84 represents the poorest quality of life outcomes. The AA-QLI consists of questions covering 3 areas of daily life: subjective symptoms, relationships, and objective signs.

CLINICAL REMISSION: As used herein, “clinical remission” refers to achieving 90% or greater hair re-growth from baseline, based on the SALT score at end of treatment (e.g., complete hair regrowth would confer a SALT score of 0).

CLINICAL RESPONSE: As used herein, “clinical response” refers to achieving 50% or greater hair re-growth from baseline, based on the SALT score at end of treatment.

ALADIN: As used herein, the “Alopecia Areata Disease Activity Index” or “ALADIN” is a three-dimensional quantitative composite gene expression score for use as a biomarker for tracking disease severity and response to treatment. See, e.g., U.S. Patent Publication 2019/0072541, which is incorporated by reference for all purposes.

AA-ASSOCIATED BIOMARKER: As used herein, the term “AA-associated biomarker” means any biological response, cell type, parameter, protein, polypeptide, enzyme, enzyme activity, metabolite, nucleic acid, carbohydrate, or other biomolecule which is present or detectable in an AA patient at a level or amount that is different from (e.g., greater than or less than) the level or amount of the marker present or detectable in a non-AA patient. The term “AA-associated biomarker” also includes a gene or gene probe known in the art which is differentially expressed in a subject with AA as compared to a subject without A. Alternatively, “AA-associated biomarker” also includes genes which are down regulated due to AA.

In some embodiments, the biomarker is assessed using histology. In some embodiments, the biomarker is assessed using RNAseq. In some embodiments, the biomarker is assessed using proteomic analysis. In some embodiments, the biomarker is assessed using enzyme-linked immunosorbent assay. In some embodiments, the biomarker is assessed using mass spectrometry. In some embodiments, the biomarker is assessed using a blood sample. In some embodiments, the biomarker will be assessed using a serum sample. In some embodiments, the biomarker will be assessed using a plasma sample. In some embodiments, the biomarker is assessed using a tissue sample. In some embodiments, the biomarker will be assessed using a punch biopsy.

In some embodiments, the biomarker is selected from Th2/IL-13, Th22/IL-22, Th1/IFN-γ, and Th17/IL-17A. In some embodiments, the biomarker is selected from IFN-γ, IL-2, IL-12, IL-13, IL-10, and IL-17. In some embodiments, the biomarker is selected from at least one of IL-2, IL-10, IL-12, IL-13, IL-17, IL-17A, IL-22, and IFN-γ. In some embodiments, the biomarker is IL-2. In some embodiments, the biomarker is IL-10. In some embodiments, the biomarker is IL-12. In some embodiments, the biomarker is IL-13. In some embodiments, the biomarker is IL-17. In some embodiments, the biomarker is IL-17A. In some embodiments, the biomarker is IL-22. In some embodiments, the biomarker is IFN-γ.

In some embodiments, the biomarker is a gene expression signature. In some embodiments, the gene expression signature comprises gene expression information of one or more of the following groups of genes: hair keratin (KRT) associated genes, cytotoxic T lymphocyte infiltration (CTL) associated genes, and interferon (IFN) associated genes. In some embodiments, the KRT-associated genes comprise DSG4, HOXC31, KRT31, KRT32, KRT33B, KRT82, PKP1 and/or PKP2. In some embodiments, the CTL-associated genes comprise CD8A, GZMB, ICOS and/or PRF1. In some embodiments, the IFN-associated genes comprise CXCL9, CXCL10, CXCL11, STAT1 and/or MX1.

In some embodiments, the AA-associated biomarker is chosen from IL-15, CCL2, CCL3, CXCL10, IL-13, CCL13, CCL17, CCL22, CCL26, CCL4, and CCL11 and the level of the biomarker is increased in the sera from individuals with AA as compared with sera from healthy patients.

In some embodiments, the AA-associated biomarker is chosen from IL-15 and eotaxin/CCL11 and the level of the biomarker is associated with SALT score.

In some embodiments, the AA-associated biomarker is chosen from scalp T_{H}2-related markers (CCL13 and IL-13) and serum T-cell/NK-cell activation marker (IL-15).

Certain embodiments relate to use of these biomarkers for monitoring disease reversal with the administration of Compound 1, or a pharmaceutically acceptable salt or as a solvate or hydrate thereof. Methods for detecting and/or quantifying such AA-associated biomarkers are known in the art; kits for measuring such AA-associated biomarkers are available from various commercial sources; and various commercial diagnostic laboratories offer services which provide measurements of such biomarkers as well.

In some embodiments, the AA-associated biomarker is a gene expression signature that is an Alopecia Areata Disease Activity Index (ALADIN). In some embodiments, the AA-associated biomarker is an Alopecia Areata Gene Signature (AAGS) comprising one or more genes set forth below.

| | |
|---|---|
| NCBI | |
| GenBank | |
| ID | Official Gene Name |

T cell activation

| | |
|---|---|
| 596 | B-cell CLL/lymphoma 2 |
| 914 | CD2 molecule |
| 915 | CD3d molecule, delta (CD3-TCR complex) |
| 920 | CD4 molecule |
| 972 | CD74 molecule, major histocompatibility complex, class II invariant chain |
| 942 | CD86 molecule |
| 925 | CD8a molecule |
| 926 | CD8b molecule |
| 10320 | IKAROS family zinc finger 1 (lkaros) |
| 8440 | NCK adaptor protein 2 |
| 1499 | catenin (cadherin-associated protein), beta 1, 88 kDa |
| 55636 | chromodomain helicase DNA binding protein 7 |
| 8320 | eomesodermin homolog (Xenopus laevis) |
| 3683 | integrin, alpha L (antigen CD11A (p180), lymphocyte function-associated antigen 1; |
| | alpha polypeptide) |
| 3684 | integrin, alpha M (complement component 3 receptor 3 subunit) |
| 3659 | interferon regulatory factor 1 |
| 3600 | interleukin 15 |
| 3936 | lymphocyte cytosolic protein 1 (L-plastin) |
| 3932 | lymphocyte-specific protein tyrosine kinase |
| 3108 | major histocompatibility complex, class II, DM alpha |
| 6693 | sialophorin |
| 387357 | thymocyte_selection pathway associated |

immune response

| | |
|---|---|
| 596 | B-cell CLL/lymphoma 2 |
| 29760 | B-cell linker |
| 23601 | C-type lectin domain family 5, member A |
| 929 | CD14 molecule |
| 9332 | CD163 molecule |
| 100133941 | CD24 molecule; CD24 molecule-like 4 |
| 972 | CD74 molecule, major histocompatibility complex, class II invariant chain |
| 9308 | CD83 molecule |
| 8832 | CD84 molecule |
| 50848 | F11 receptor |
| 2268 | Gardner-Rasheed feline sarcoma viral (v-fgr) oncogene homolog |
| 10866 | HLA complex P5 |
| 150372 | NFAT activating protein with ITAM motif 1 |
| 25939 | SAM domain and HD domain 1 |
| 50852 | T cell receptor associated transmembrane adaptor 1 |
| 7078 | TIMP metallopeptidase inhibitor 3 |
| 7305 | TYRO protein tyrosine kinase binding protein |
| 11326 | V-set and immunoglobulin domain containing 4 |
| 84632 | actin filament associated protein 1-like 2 |
| 90 | activin A receptor, type I |
| 199 | allograft inflammatory factor 1 |
| 197 | alpha-2-HS-glycoprotein |
| 60489 | apolipoprotein B mRNA editing enzyme, catalytic polypeptide-like 3G |
| 80833 | apolipoprotein L, 3 |
| 650 | bone morphogenetic protein 2 |
| 9435 | carbohydrate (N-acetylglucosamine-6-O)_sulfotransferase 2 |
| 1508 | cathepsin B |
| 6357 | chemokine (C-C motif) ligand 13 |
| 6362 | chemokine (C-C motif) ligand 18 (pulmonary and activation-regulated) |
| 6351 | chemokine (C-C motif) ligand 4 |
| 6352 | chemokine (C-C motif) ligand 5 |
| 6355 | chemokine (C-C motif) ligand 8 |
| 1230 | chemokine (C-C motif) receptor 1 |
| 1234 | chemokine (C-C motif) receptor 5 |
| 3627 | chemokine (C-X-C motif) ligand 10 |
| 4283 | chemokine (C-X-C motif) ligand 9 |
| 4261 | class II, major histocompatibility complex, transactivator |
| 713 | complement component 1, q subcomponent, B chain |
| 714 | complement component 1, q subcomponent, C chain |
| 1755 | deleted in malignant brain tumors 1 |
| 8456 | forkhead box N1 |
| 3055 | hemopoietic cell kinase |
| 8347, | histone cluster 1, H2bi; histone cluster 1, H2bg; histone |
| 8343, | |
| 8346, | cluster 1, H2be; histone cluster 1, H2bf; histone cluster 1, |
| 8344, | |
| 8339 | H2bc |
| 3399 | inhibitor of DNA binding 3, dominant negative helix-loop-helix protein |
| 3683 | integrin, alpha L (antigen CD11A (p180), lymphocyte function-associated antigen 1; |
| | alpha polypeptide) |
| 3689 | integrin, beta 2 (complement component 3 receptor 3 and 4 subunit) |
| 3694 | integrin, beta 6 |
| 64135 | interferon induced with helicase C domain 1 |
| 338376 | interferon, epsilon |
| 3600 | interleukin 15 |
| 9235 | interleukin 32 |
| 3579 | interleukin 8 receptor, beta |
| 3822 | killer cell lectin-like receptor subfamily C, member 2 |
| 3988 | lipase A, lysosomal acid, cholesterol esterase |
| 58530 | lymphocyte antigen 6 complex, locus G6F; lymphocyte antigen 6 complex, locus G6D |
| 3107, | major histocompatibility complex, class I, C; major histocompatibility complex, class |
| 3106 | I, B |
| 4332 | myeloid cell nuclear differentiation antigen |
| 4542 | myosin IF |
| 5551 | perforin 1 (pore forming protein) |
| 30814 | phospholipase A2, group IIE |
| 5341 | pleckstrin |
| 10544 | protein C receptor, endothelial (EPCR) |
| 5265 | serpin peptidase inhibitor, clade A (alpha-1 antiproteinase, antitrypsin), member 1 |
| 12 | serpin peptidase inhibitor, clade A (alpha-1 antiproteinase, antitrypsin), member 3 |
| 6614 | sialic acid binding Ig-like lectin 1, sialoadhesin |
| 6693 | sialophorin |
| 6469 | sonic hedgehog homolog (Drosophila) |
| 7057 | thrombospondin 1 |
| 7096 | toll-like receptor 1 |
| 7042 | transforming growth factor, beta 2 |
| 6890 | transporter 1, ATP-binding cassette,_sub-family B (MDR/TAP) |
| 7133 | tumor necrosis factor receptor_superfamily, member 1B |
| 7534 | tyrosine 3-monooxygenase/tryptophan 5-monooxygenase activation protein, zeta |
| | polypeptide |

plasma membrane

| | |
|---|---|
| 8909 | 26_serine protease |
| 417 | ADP-ribosyltransferase 1 |
| 24 | ATP-binding cassette,_sub-family A (ABC1), member 4 |
| 5243 | ATP-binding cassette,_sub-family B (MDR/TAP), member 1 |
| 5244 | ATP-binding cassette,_sub-family B (MDR/TAP), member 4 |
| 9619 | ATP-binding cassette, sub-family G (WHITE), member 1 |
| 29760 | B-cell linker |
| 598 | BCL2-like 1 |
| 23601 | C-type lectin domain family 5, member A |
| 160365 | C-type lectin-like 1 |
| 135228 | CD109 molecule |
| 929 | CD14 molecule |
| 9332 | CD163 molecule |
| 911 | CD1c molecule |
| 914 | CD2 molecule |
| 30835 | CD209 molecule |
| 100133941 | CD24 molecule; CD24 molecule-like 4 |
| 915 | CD3d molecule, delta (CD3-TCR complex) |
| 920 | CD4 molecule |
| 1043 | CD52 molecule |
| 963 | CD53 molecule |
| 972 | CD74 molecule, major histocompatibility complex, class II invariant chain |
| 3732 | CD82 molecule |
| 9308 | CD83 molecule |
| 8832 | CD84 molecule |
| 942 | CD86 molecule |
| 925 | CD8a molecule |
| 926 | CD8b molecule |
| 10225 | CD96 molecule |
| 56882 | CDC42_small effector 1 |
| 30845 | EH-domain containing 3 |
| 1969 | EPH receptor A2 |
| 2048 | EPH receptor B2 |
| 50848 | F11 receptor |
| 22844 | FERM and PDZ domain containing 1 |
| 2205 | Fc fragment of IgE, high affinity I, receptor for; alpha polypeptide |
| 2212 | Fc fragment of IgG, low affinity IIa, receptor (CD32) |
| 2213 | Fc fragment of IgG, low affinity IIb, receptor (CD32); Fc fragment of IgG, low |
| | affinity IIc, receptor for (CD32) |
| 166647 | G protein-coupled receptor 125 |
| 23432 | G protein-coupled receptor 161 |
| 1880 | G protein-coupled receptor 183 |
| 55507 | G protein-coupled receptor, family C, group 5, member D |
| 3927 | LIM and SH3 protein 1 |
| 130576 | LY6/PLAUR domain containing 6B |
| 65108 | MARCKS-like 1 |
| 3071 | NCK-associated protein 1-like |
| 150372 | NFAT activating protein with ITAM motif 1 |
| 4864 | Niemann-Pick disease, type C1 |
| 5754 | PTK7 protein tyrosine kinase 7 |
| 376267 | RAB15, member RAS onocogene family |
| 22931 | RAB18, member RAS oncogene family |
| 285613 | RELT-like 2 |
| 56963 | RGM domain family, member A |
| 23504 | RIMS binding protein 2 |
| 10900 | RUN domain containing 3A |
| 6016 | Ras-like without CAAX 1 |
| 51458 | Rh family, C glycoprotein |
| 30011 | SH3-domain kinase binding protein 1 |
| 4092 | SMAD family member 7 |
| 8869 | ST3 beta-galactoside alpha-2,3-sialyltransferase 5 |
| 6461 | Src homology 2 domain containing adaptor protein B |
| 50852 | T cell receptor associated transmembrane adaptor 1 |
| 28639 | T cell receptor beta variable 19; T cell receptor beta constant 1 |
| 6967 | T cell receptor gamma locus; T cell receptor gamma constant 2 |
| 445347 | TCR gamma alternate reading frame protein; T cell receptor gamma variable 9; T cell |
| | receptor gamma constant 1 |
| 7305 | TYRO protein tyrosine kinase binding protein |
| 11326 | V-set and immunoglobulin domain containing 4 |
| 65266 | WNK lysine deficient protein kinase 4 |
| 10152 | abl interactor 2 |
| 90 | activin A receptor, type I |
| 120425 | adhesion molecule, interacts with CXADR antigen 1 |
| 199 | allograft inflammatory factor 1 |
| 83543 | allograft inflammatory factor 1-like |
| 351 | amyloid beta (A4) precursor protein |
| 56899 | ankyrin repeat and sterile alpha motif domain containing 1B |
| 83464 | anterior pharynx defective 1 homolog B (C. elegans) |
| 54796 | basonuclin 2 |
| 144453 | bestrophin 3 |
| 685 | Betacellulin |
| 1952 | cadherin, EGF LAG_seven-pass G-type receptor 2 (flamingo homolog, Drosophila) |
| 776 | calcium channel, voltage-dependent, L type, alpha 1D subunit |
| 8913 | calcium channel, voltage-dependent, T type, alpha 1G subunit |
| 27092 | calcium channel, voltage-dependent, gamma subunit 4 |
| 800 | caldesmon 1 |
| 768 | carbonic anhydrase IX |
| 1499 | catenin (cadherin-associated protein), beta 1, 88 kDa |
| 1500 | catenin (cadherin-associated protein), delta 1 |
| 1501 | catenin (cadherin-associated protein), delta 2 (neural plakophilin-related arm-repeat |
| | protein) |
| 1508 | cathepsin B |
| 1230 | chemokine (C-C motif) receptor 1 |
| 1234 | chemokine (C-C motif) receptor 5 |
| 25932 | chloride intracellular channel 4 |
| 1464 | chondroitin sulfate proteoglycan 4 |
| 23562 | claudin 14 |
| 1436 | colony_stimulating factor 1 receptor |
| 594855 | complexin 3 |
| 1525 | coxsackie virus and adenovirus receptor pseudogene 2; coxsackie virus and |
| | adenovirus receptor |
| 26999 | cytoplasmic FMR1 interacting protein 2 |
| 1824 | desmocollin 2 |
| 1830 | desmoglein 3 (pemphigus vulgaris antigen) |
| 147409 | desmoglein 4 |
| 55740 | enabled homolog (Drosophila) |
| 30816 | endogenous retroviral family W, env(C7), member 1 |
| 1946 | ephrin-A5 |
| 2099 | estrogen receptor 1 |
| 2260 | fibroblast growth factor receptor 1 |
| 23767 | fibronectin leucine rich transmembrane protein 3 |
| 54751 | filamin binding LIM protein 1 |
| 2323 | fms-related tyrosine kinase 3 ligand |
| 2350 | folate receptor 2 (fetal) |
| 342184 | formin 1 |
| 7976 | frizzled homolog 3 (Drosophila) |
| 8323 | frizzled homolog 6 (Drosophila) |
| 8324 | frizzled homolog 7 (Drosophila) |
| 2523 | fucosyltransferase 1 (galactoside 2-alpha-L-fucosyltransferase, H blood group) |
| 2554 | gamma-aminobutyric acid (GABA) A receptor, alpha 1 |
| 2561 | gamma-aminobutyric acid (GABA) A receptor, beta 2 |
| 2700 | gap junction protein, alpha 3, 46 kDa |
| 2706 | gap junction protein, beta 2, 26 kDa |
| 10804 | gap junction protein, beta 6, 30 kDa |
| 125111 | gap junction protein, delta 3, 31.9 kDa |
| 342035 | Gliomedin |
| 2892 | glutamate receptor, ionotrophic, AMPA 3 |
| 3001 | granzyme A (granzyme 1, cytotoxic T-lymphocyte-associated_serine esterase 3) |
| 3002 | granzyme B (granzyme 2, cytotoxic T-lymphocyte-associated_serine esterase 1) |
| 2774 | guanine nucleotide binding protein (G protein), alpha activating activity polypeptide, |
| | olfactory type |
| 2782 | guanine nucleotide binding protein (G protein), beta polypeptide 1 |
| 115362 | guanylate binding protein 5 |
| 64399 | hedgehog interacting protein |
| 9456 | homer homolog 1 (Drosophila) |
| 9455 | homer homolog 2 (Drosophila) |
| 3683 | integrin, alpha L (antigen CD11A (p180), lymphocyte function-associated antigen 1; |
| | alpha polypeptide) |
| 3684 | integrin, alpha M (complement component 3 receptor 3 subunit) |
| 3687 | integrin, alpha X (complement component 3 receptor 4 subunit) |
| 3689 | integrin, beta 2 (complement component 3 receptor 3 and 4 subunit) |
| 3694 | integrin, beta 6 |
| 3587 | interleukin 10 receptor, alpha |
| 3594 | interleukin 12 receptor, beta 1 |
| 3600 | interleukin 15 |
| 3561 | interleukin 2 receptor, gamma (severe combined immunodeficiency) |
| 3579 | interleukin 8 receptor, beta |
| 182 | jagged 1 (Alagille_syndrome) |
| 58494 | junctional adhesion molecule 2 |
| 3821 | killer cell lectin-like receptor_subfamily C, member 1 |
| 3822 | killer cell lectin-like receptor_subfamily C, member 2 |
| 22914 | killer cell lectin-like receptor_subfamily K, member 1 |
| 8549 | leucine-rich repeat-containing G protein-coupled receptor 5 |
| 3977 | leukemia inhibitory factor receptor alpha |
| 58530 | lymphocyte antigen 6 complex, locus G6F; lymphocyte antigen 6 complex, locus G6D |
| 3936 | lymphocyte cytosolic protein 1 (L-plastin) |
| 3932 | lymphocyte-specific protein tyrosine kinase |
| 4033 | lymphoid-restricted membrane protein |
| 9170 | lysophosphatidic acid receptor 2 |
| 23566 | lysophosphatidic acid receptor 3 |
| 3107, | major histocompatibility complex, class I, C; major histocompatibility complex, class |
| 3106 | I, B |
| 3134 | major histocompatibility complex, class I, F |
| 3108 | major histocompatibility complex, class II, DM alpha |
| 3109 | major histocompatibility complex, class II, DM beta |
| 3111 | major histocompatibility complex, class II, DO alpha |
| 3113 | major histocompatibility complex, class II, DP alpha 1 |
| 3119 | major histocompatibility complex, class II, DQ beta 1; similar to major |
| | histocompatibility complex, class II, DQ beta 1 |
| 4118 | mal, T-cell differentiation protein |
| 4360 | mannose receptor, C type 1 |
| 55686 | Melanoregulin |
| 154043, | membrane associated guanylate kinase, WW and PDZ domain containing 1; CNKSR |
| 9223 | family member 3 |
| 23499 | microtubule-actin crosslinking factor 1 |
| 9053 | microtubule-associated protein 7 |
| 4128 | monoamine oxidase A |
| 4155 | myelin basic protein |
| 8828 | neuropilin 2 |
| 4846 | nitric oxide_synthase 3 (endothelial cell) |
| 123264 | organic_solute transporter beta |
| 29780 | parvin, beta |
| 64098 | parvin, gamma |
| 5551 | perforin 1 (pore forming protein) |
| 5141 | phosphodiesterase 4A, cAMP-specific (phosphodiesterase E2 dunce homolog, |
| | Drosophila) |
| 27445 | piccolo (presynaptic cytomatrix protein) |
| 5317 | plakophilin 1 (ectodermal dysplasia/skin fragility syndrome);_similar to plakophilin 1 |
| | isoform 1a |
| 11187 | plakophilin 3 |
| 5341 | Pleckstrin |
| 23362 | pleckstrin and Sec7 domain containing 3 |
| 5362 | plexin A2 |
| 5818 | poliovirus receptor-related 1 (herpesvirus entry mediator C) |
| 81607 | poliovirus receptor-related 4 |
| 200845 | potassium channel tetramerisation domain containing 6 |
| 3784 | potassium voltage-gated channel, KQT-like subfamily, member 1 |
| 56937 | prostate transmembrane protein, androgen induced 1 |
| 10544 | protein C receptor, endothelial (EPCR) |
| 5579 | protein kinase C, beta |
| 5587 | protein kinase D1 |
| 26051 | protein phosphatase 1, regulatory (inhibitor) subunit 16B |
| 5099 | protocadherin 7 |
| 53829 | purinergic receptor P2Y, G-protein coupled, 13 |
| 9934 | purinergic receptor P2Y, G-protein coupled, 14 |
| 54509 | ras homolog gene family, member F (in filopodia) |
| 9699 | regulating_synaptic membrane exocytosis 2 |
| 9783 | regulating_synaptic membrane exocytosis 3 |
| 6248 | regulatory_solute carrier protein, family 1, member 1 |
| 22800 | related RAS viral (r-ras) oncogene homolog 2; similar to related RAS viral (r-ras) |
| | oncogene homolog 2 |
| 6404 | selectin P ligand |
| 64218 | sema domain, immunoglobulin domain (Ig), transmembrane domain (TM) and_short |
| | cytoplasmic domain, (semaphorin) 4A |
| 6614 | sialic acid binding Ig-like lectin 1, sialoadhesin |
| 89790 | sialic acid binding Ig-like lectin 10 |
| 6693 | sialophorin |
| 140885 | signal-regulatory protein alpha |
| 55423 | signal-regulatory protein gamma |
| 6504 | signaling lymphocytic activation molecule family member 1 |
| 4301 | similar to Afadin (Protein AF-6); myeloid/lymphoid or mixed-lineage leukemia |
| | (trithorax homolog, Drosophila); translocated to, 4 |
| 57228 | small trans-membrane and glycosylated protein |
| 6509 | solute carrier family 1 (glutamate/neutral amino acid transporter), member 4 |
| 6511 | solute carrier family 1 (high affinity aspartate/glutamate transporter), member 6 |
| 10723 | solute carrier family 12 (potassium/chloride transporters), member 7 |
| 9120 | solute carrier family 16, member 6 (monocarboxylic acid transporter 7);_similar |
| | to_solute carrier family 16, member 6 |
| 220963 | solute carrier family 16, member 9 (monocarboxylic acid transporter 9) |
| 6575 | solute carrier family 20 (phosphate transporter), member 2 |
| 28965 | solute carrier family 27 (fatty acid transporter), member 6 |
| 10991 | solute carrier family 38, member 3 |
| 30061 | solute carrier family 40 (iron-regulated transporter), member 1 |
| 200010 | solute carrier family 5 (sodium/glucose cotransporter), member 9 |
| 11254 | solute carrier family 6 (amino acid transporter), member 14 |
| 55117 | solute carrier family 6 (neutral amino acid transporter), member 15 |
| 23428 | solute carrier family 7 (cationic amino acid transporter, y+ system), member 8 |
| 23657 | solute carrier family 7, (cationic amino acid transporter, y+ system) member 11 |
| 6751 | somatostatin receptor 1 |
| 6752 | somatostatin receptor 2 |
| 6469 | sonic hedgehog homolog (Drosophila) |
| 6272 | sortilin 1 |
| 124460 | sorting nexin 20 |
| 2040 | stomatin |
| 6854 | synapsin 11 |
| 54843 | synaptotagmin-like 2 |
| 117178 | synovial_sarcoma, X breakpoint 2 interacting protein |
| 7057 | thrombospondin 1 |
| 6915 | thromboxane A2 receptor |
| 7096 | toll-like receptor 1 |
| 7039 | transforming growth factor, alpha |
| 7053 | transglutaminase 3 (E polypeptide, protein-glutamine-gamma-glutamyltransferase) |
| 140803 | transient receptor potential cation channel,_subfamily M, member 6 |
| 4071 | transmembrane 4 L six family member 1 |
| 6890 | transporter 1, ATP-binding cassette, sub-family B (MDR/TAP) |
| 10381 | tubulin, beta 3; melanocortin 1 receptor (alpha melanocyte |
| | stimulating hormone receptor) |
| 8795 | tumor necrosis factor receptor superfamily, member 10b |
| 51330 | tumor necrosis factor receptor_superfamily, member 12A |
| 7133 | tumor necrosis factor receptor_superfamily, member 1B |
| 7126 | tumor necrosis factor, alpha-induced protein 1 (endothelial) |
| 94015 | tweety homolog 2 (Drosophila) |
| 5412 | ubiquitin-like 3 |
| 673 | v-raf murine_sarcoma viral oncogene homolog B1 |
| 6843 | vesicle-associated membrane protein 1 (synaptobrevin 1) |

antigen presentation

| | |
|---|---|
| 920 | CD4 molecule |
| 972 | CD74 molecule, major histocompatibility complex, class II invariant chain |
| 925 | CD8a molecule |
| 926 | CD8b molecule |
| 1508 | cathepsin B |
| 1520 | cathepsin S |
| 4261 | class II, major histocompatibility complex, transactivator |
| 3306 | heat shock 70 kDa protein 2 |
| 3821 | killer cell lectin-like receptor subfamily C, member 1 |
| 3822 | killer cell lectin-like receptor subfamily C, member 2 |
| 3107, | major histocompatibility complex, class I, C; major histocompatibility complex, class |
| 3106 | I, B |
| 3134 | major histocompatibility complex, class I, F |
| 3108 | major histocompatibility complex, class II, DM alpha |
| 3109 | major histocompatibility complex, class II, DM beta |
| 3111 | major histocompatibility complex, class II, DO alpha |
| 3113 | major histocompatibility complex, class II, DP alpha 1 |
| 3119 | major histocompatibility complex, class II, DQ beta 1; similar to major |
| | histocompatibility complex, class II, DQ beta 1 |
| 2923 | protein disulfide isomerase family A, member 3 |
| 5993 | regulatory factor X, 5 (influences HLA class II expression) |
| 6890 | transporter 1, ATP-binding cassette, sub-family B (MDR/TAP) |

hair cycle/hair follicle dev/epidermis dev

| | |
|---|---|
| 596 | B-cell CLL/lymphoma 2 |
| 8538 | BARX homeobox 2 |
| 2001 | E74-like factor 5 (ets domain transcription factor) |
| 646 | basonuclin 1 |
| 1499 | catenin (cadherin-associated protein), beta 1, 88 kDa |
| 1474 | cystatin ELM |
| 2068 | excision repair cross-complementing rodent repair |
| | deficiency, complementation group 2 |
| 2171 | fatty acid binding protein 5-like 2; fatty acid binding protein 5 (psoriasis-associated); |
| | fatty acid binding protein 5-like 8; fatty acid binding protein 5-like 7; fatty acid |
| | binding protein 5-like 9 |
| 2304 | forkhead box E1 (thyroid transcription factor 2) |
| 8456 | forkhead box N1 |
| 3229 | homeobox C13 |
| 182 | jagged 1 (Alagille syndrome) |
| 3868 | keratin 16; keratin type 16-like |
| 342574 | keratin 27 |
| 3881 | keratin 31 |
| 3882 | keratin 32 |
| 3885 | keratin 34 |
| 3854 | keratin 6B |
| 3889 | keratin 83 |
| 3891 | keratin 85 |
| 3846 | keratin associated protein 5-9 |
| 51176 | lymphoid enhancer-binding factor 1 |
| 55686 | melanoregulin |
| 5017 | ovo-like 1(Drosophila) |
| 864 | runt-related transcription factor 3 |
| 6469 | sonic hedgehog homolog (Drosophila) |
| 7042 | transforming growth factor, beta 2 |
| 7053 | transglutaminase 3 (E polypeptide, protein-glutamine-gamma-glutamyltransferase) |

PHARMACEUTICAL COMPOSITION: As used here, “pharmaceutical composition” means a composition comprising at least one active ingredient, such as Compound 1, including but not limited to, salts, solvates, and hydrates of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

HYDRATE: As used herein, “hydrate” means a compound of the invention or a salt thereof, that further includes a stoichiometric or non-stoichiometric amount of water bound by non-covalent intermolecular forces.

SOLVATE: As used herein, “solvate” means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66: 1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, DE), Avantium Technologies (Amsterdam) and Aptuit (Greenwich, CT).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions, and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1 or a pharmaceutically acceptable salt, solvate, or hydrate thereof can be separated into two methods; one method reciting prescribing Compound 1 or a pharmaceutically acceptable salt, solvate, or hydrate thereof and the other method reciting administering Compound 1 or a pharmaceutically acceptable salt, solvate, or hydrate thereof. In addition, for example, a method that recites prescribing Compound 1 or a pharmaceutically acceptable salt, solvate, or hydrate thereof and a separate method of the invention reciting administering Compound 1 or a pharmaceutically acceptable salt, solvate, or hydrate thereof can be combined into a single method reciting prescribing and/or administering Compound 1 or a pharmaceutically acceptable salt, solvate, or hydrate thereof.

Provided is a method of treating alopecia areata in an individual in need thereof comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt, hydrate, or solvate thereof.

In some embodiments, the individual has severe alopecia areata. In some embodiments, the individual has moderate alopecia areata. In some embodiments, the individual has mild alopecia areata.

In some embodiments, the individual has diffuse alopecia areata. In some embodiments, the individual has alopecia areata monolocularis. In some embodiments, the individual has alopecia areata multilocularis. In some embodiments, the individual has ophiasis. In some embodiments, the individual has alopecia areata barbae. In some embodiments, the individual has alopecia areata totalis. In some embodiments, the individual has is alopecia areata universalis.

In some embodiments, the individual is assessed for AA severity. In some embodiments, the individual is assessed using SALT I. In some embodiments, the individual is assessed using SALT II. In some embodiments, the individual is assessed using a patient-reported outcome (PRO) measurement. In some embodiments, the individual is assessed for quality of life. In some embodiments, the individual is assessed using a questionnaire. In some embodiments, the individual is assessed using the Alopecia Areata Symptom Impact Scale (AASIS). See Mendoza T R, Osei J, Duvic M. The utility and validity of the Alopecia Areata Symptom Impact Scale in measuring disease-related symptoms and their effect on functioning. J. Investig Dermatol Symp Proc. 2018; 19(1): S41-S46. In some embodiments, the individual is assessed using the Alopecia Areata-Related Quality of Life (AA-QLI) questionnaire. See Fabbrocini G, Panariello L, De Vita V, et al. Quality of life in alopecia areata: A disease-specific questionnaire. J Eur Acad Dermatol Venereol. 2013; 27(3): e276-281. In some embodiments, the individual is assessed using the Skindex-16 measure of quality of life. See Chren M M. The Skindex instruments to measure the effects of skin disease on quality of life. Dermatol Clin. 2012; 30(2): 231-236. In some embodiments, the individual is assessed using the Dermatology Life Quality Index (DLQI). See Finlay A Y, Khan G K. Dermatology Life Quality Index (DLQI)—a simple practical measure for routine clinical use. Clin Exp Dermatol. 1994; 19(3): 210-216; Basra M K A, Salek M S, Camilleri L, Sturkey R, Finlay A Y. Determining the minimal clinically important difference and responsiveness of the Dermatology Life Quality Index (DLQI): Further data. Dermatology. 2015; 230(1): 27-33. In some embodiments, the fingernails and/or toenails of the individual are assessed (e.g., for dents, white spots, or roughness).

In some embodiments, the individual in need of treatment has a SALT score of at least 30, 35, 40, 45, 50, 55, 60, 65, 70, 75, 80, 85, 90, 95, or 100. In some embodiments, the individual has greater than or equal to 50% scalp hair loss. In some embodiments, the method is therapeutically effective to achieve at least a 50% improvement from the individual's baseline Severity of Alopecia Tool (SALT) score. In some embodiments, the method is therapeutically effective to achieve at least a 50% improvement from the individual's baseline SALT score in a period of time of at least about 24 weeks. In some embodiments, the method is therapeutically effective to achieve at least a 50% improvement from the individual's baseline SALT score in a period of time of about 24 weeks. In some embodiments, the method is therapeutically effective to achieve at least a 50% improvement from the individual's baseline SALT score in a period of time of at least about 52 weeks. In some embodiments, the method is therapeutically effective to achieve at least a 50% improvement from the individual's baseline SALT score in a period of time of about 52 weeks. In some embodiments, the method is therapeutically effective to achieve at least a 50% improvement from the individual's baseline SALT score in a period of time of at least about 4, 8, 12, or 20 weeks. In some embodiments, the method is therapeutically effective to achieve at least a 30% improvement from the individual's baseline Severity of Alopecia Tool (SALT) score. In some embodiments, the method is therapeutically effective to achieve at least a 30% improvement from the individual's baseline SALT score in a period of time of at least about 24 weeks. In some embodiments, the method is therapeutically effective to achieve at least a 30% improvement from the individual's baseline SALT score in a period of time of about 52 weeks. In some embodiments, the method is therapeutically effective to achieve at least a 30% improvement from the individual's baseline SALT score in a period of time of at least about 4, 8, 12, or 20 weeks. In some embodiments, the method is therapeutically effective to achieve at least a 75% improvement from the individual's baseline Severity of Alopecia Tool (SALT) score. In some embodiments, the method is therapeutically effective to achieve at least a 75% improvement from the individual's baseline SALT score in a period of time of at least about 24 weeks. In some embodiments, the method is therapeutically effective to achieve at least a 75% improvement from the individual's baseline SALT score in a period of time of about 52 weeks. In some embodiments, the method is therapeutically effective to achieve at least a 75% improvement from the individual's baseline SALT score in a period of time of at least about 4, 8, 12, or 20 weeks.

In some embodiments, the method is therapeutically effective to achieve at least a 30% improvement from the individual's baseline AASIS score. In some embodiments, the method is therapeutically effective to achieve at least a 30% improvement from the individual's baseline AASIS score in a period of time of about 24 weeks. In some embodiments, the method is therapeutically effective to achieve at least a 30% improvement from the individual's baseline AASIS score in a period of time of at least about 52 weeks. In some embodiments, the method is therapeutically effective to achieve at least a 50% improvement from the individual's baseline AASIS score. In some embodiments, the method is therapeutically effective to achieve at least a 50% improvement from the individual's baseline AASIS score in a period of time of about 24 weeks. In some embodiments, the method is therapeutically effective to achieve at least a 50% improvement from the individual's baseline AASIS score in a period of time of at least about 52 weeks.

In some embodiments, the method is therapeutically effective to achieve at least a 30% improvement from the individual's baseline AA-QLI score. In some embodiments, the method is therapeutically effective to achieve at least a 30% improvement from the individual's baseline AA-QLI score in a period of time of about 24 weeks. In some embodiments, the method is therapeutically effective to achieve at least a 30% improvement from the individual's baseline AA-QLI score in a period of time of at least about 52 weeks. In some embodiments, the method is therapeutically effective to achieve at least a 50% improvement from the individual's baseline AA-QLI score. In some embodiments, the method is therapeutically effective to achieve at least a 50% improvement from the individual's baseline AA-QLI score in a period of time of about 24 weeks. In some embodiments, the method is therapeutically effective to achieve at least a 50% improvement from the individual's baseline AA-QLI score in a period of time of at least about 52 weeks.

According to some embodiments, the method is therapeutically effective to reduce hair shedding in an individual diagnosed with alopecia areata. In some embodiments, the method is therapeutically effective to prevent hair loss in an individual diagnosed with alopecia areata. According to some embodiments, the method is therapeutically effective to induce hair growth in an individual diagnosed with alopecia areata. In some embodiments, the method is therapeutically effective to induce hair regrowth in an individual diagnosed with alopecia areata. According to some embodiments, the method is therapeutically effective to induce hair regrowth on at least 30% of the scalp of an individual suffering from alopecia areata. In some embodiments, the method is therapeutically effective to induce hair regrowth on at least 50% of the scalp of an individual suffering from alopecia areata. According to some embodiments, the method is therapeutically effective to induce hair regrowth on at least 75% of the scalp of an individual suffering from alopecia areata.

In some embodiments, the method further comprises detecting in the individual an AA-associated biomarker. In some embodiments, prior to administering Compound 1, or a pharmaceutically acceptable salt, hydrate, or solvate thereof, to the individual, the method further comprises selecting the individual based on a level of an AA-associated biomarker. In some embodiments, administration of Compound 1, or a pharmaceutically acceptable salt, hydrate, or solvate thereof, results in a change in a level of an AA-associated biomarker in the individual. In some embodiments, the AA-associated biomarker is indicative of severity of the alopecia areata. In some embodiments, the AA-associated biomarker is indicative of the propensity of the individual to respond to treatment with Compound 1, or a pharmaceutically acceptable salt, hydrate, or solvate thereof.

In some embodiments, the individual shows elevated levels of one or more AA-associated biomarkers. In some embodiments, the administration of Compound 1, or a pharmaceutically acceptable salt, hydrate, or solvate thereof, results in reducing the level of an AA-associated biomarker in the individual.

In some embodiments, the pharmaceutical dosage form is administered once daily to the individual.

In some embodiments, the individual is administered an amount equivalent to about 0.5 to about 5.0 mg of Compound 1. In some embodiments, the individual is administered an amount equivalent to 1 mg of Compound 1. In some embodiments, the individual is administered an amount equivalent to 1.5 mg of Compound 1. In some embodiments, the individual is administered an amount equivalent to 2 mg of Compound 1. In some embodiments, the individual is administered an amount equivalent to 2.25 mg of Compound 1. In some embodiments, the individual is administered an amount equivalent to 2.5 mg of Compound 1. In some embodiments, the individual is administered an amount equivalent to 2.75 mg of Compound 1. In some embodiments, the individual is administered an amount equivalent to 3 mg of Compound 1.

In some embodiments, the individual is administered Compound 1 or a pharmaceutically acceptable salt thereof at least one month, such as one month, two months, three months, four months, etc. In some embodiments, the individual is administered Compound 1 or a pharmaceutically acceptable salt thereof for least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, etc. In some embodiments, the period is indefinite, e.g., chronic administration.

In some embodiments, the individual is administered an amount equivalent to 2 mg of Compound 1 for a first time period and subsequently an amount equivalent to 3 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is indefinite, e.g., chronic administration.

In some embodiments, the individual is administered an amount equivalent to 3 mg of Compound 1 for a first time period and subsequently an amount equivalent to 2 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is indefinite, e.g., chronic administration.

In some embodiments, the dosage form is administered with titration. In some embodiments, the standard dose is administered without titration. In some embodiments, the standard dose is administered without titration; and the individual does not experience a severe related adverse event. In some embodiments, the standard dose is administered without requiring titration to avoid first-dose effect seen with other S1P receptor modulators.

In some embodiments, the dosage form is administered under fasted conditions. In some embodiments, the dosage form is administered under fed conditions.

In some embodiments, the method is non-gender specific.

In some embodiments, Compound 1, or a pharmaceutically acceptable salt thereof, is administered in combination with a second therapeutic agent or therapy. In some embodiments, the second therapeutic agent or therapy is for the treatment of an autoimmune disease other than AA. In some embodiments, the second therapeutic agent or therapy is for the treatment of atopic dermatitis, allergic rhinitis, vitiligo, and/or psoriasis.

In some embodiments, the second therapeutic agent or therapy for the treatment of atopic dermatitis is chosen from a skin emollient like petroleum jelly, topical steroids, oral antihistamines, and/or antibiotics.

In some embodiments, the second therapeutic agent or therapy for the treatment of allergic rhinitis is chosen from antihistamines, decongestants, eye drops and/or nasal sprays to relieve itchiness and other allergy-related symptoms, and/or immunotherapy or allergy shots.

In some embodiments, the second therapeutic agent or therapy for the treatment of vitiligo is chosen from sunscreen, topical corticosteroid cream, topical oxsoralen, mini grafting, and/or PUVA photochemotherapy.

In some embodiments, the second therapeutic agent or therapy for the treatment of psoriasis is chosen from Vitamin D analogues, anthralin, retinoids, calcineurin inhibitors, salicylic acid, coal tar, moisturizers, phototherapy, methotrexate, cyclosporine, thioguanine, hydroxyurea, and biologics such as etanercept (Enbrel), infliximab (Remicade), adalimumab (Humira), ustekinumab (Stelara), golimumab (Simponi), apremilast (Otezla), secukinumab (Cosentyx) and ixekizumab (Taltz).

In some embodiments, the individual is not administered a steroid, such as a corticosteroid, a topical steroid and/or a topical corticosteroid cream. In some embodiments, the individual is not administered a topical, intralesional, or systemic corticosteroid. In some embodiments, the individual is not administered a systemic glucocorticoid. In some embodiments, the individual is not administered a topical calcineurin inhibitor. In some embodiments, the individual is not administered minoxidil, such as topical or oral minoxidil. In some embodiments, the individual is not administered bimatoprost, such as topical bimatoprost. In some embodiments, the individual is not administered a topical prescription medication for AA. In some embodiments, the individual is not administered a systemic glucocorticoid. In some embodiments, the individual is not administered immunoglobulin or blood products. In some embodiments, the individual is not administered a systemic immunosuppressive and/or immunomodulating drug, such as cyclosporine, azathioprine, and/or methotrexate. In some embodiments, the individual is not administered a JAK inhibitor, such as a topical or an oral JAK inhibitor. In some embodiments, the individual is not administered a biologic, such as dupilumab (Dupixent), etanercept (Enbrel), infliximab (Remicade), adalimumab (Humira), ustekinumab (Stelara), golimumab (Simponi), apremilast (Otezla), secukinumab (Cosentyx) and/or ixekizumab (Taltz). In some embodiments, the individual is not administered a cell-depleting agent, such as rituximab. In some embodiments, the individual is not administered the foregoing therapeutic agents or therapies prior to administration of Compound 1, or a pharmaceutically acceptable salt, hydrate, or solvate thereof. In some embodiments, the individual is not co-administered the foregoing therapeutic agents or therapies during the administration of Compound 1, or a pharmaceutically acceptable salt, hydrate, or solvate thereof.

In some embodiments, the second therapeutic agent or therapy is for the treatment of anxiety and/or depression. In some embodiments, the second therapeutic agent or therapy for the treatment of anxiety and/or depression is chosen from psychotherapy, antidepressants, buspirone, and benzodiazepines.

In some embodiments, an individual administered Compound 1, or a pharmaceutically acceptable salt, hydrate, or solvate thereof, was previously administered at least one therapeutic agent or therapy for the treatment of AA. In some embodiments, the at least one previously administered therapeutic agent or therapy for the treatment of AA is chosen from corticosteroids, topical immunotherapy, minoxidil, anthralin, squaric acid dibutylester, and diphencyprone.

In some embodiments, the at least one therapeutic agent or therapy for the treatment of AA is a JAK inhibitor. As used herein, a “JAK inhibitor” refers to a compound that interacts with a Jak 1/Jak2/Jak3/Tyk2/STAT1/STAT2/STAT3/STAT4/STAT5a/STAT5b/STAT6/OSM/gp 130/LIFR/OSM-Rβ gene or a Jak1/Jak2/Jak3/Tyk2/STAT1/STAT2/STAT3/ STAT4/STAT5a/STAT5b/STAT6/OSM/gp130-/LIFR/OSM-Rβ protein or polypeptide and inhibits its activity and/or its expression. The compound can decrease the activity or expression of a protein encoded by Jak1/Jak2/Jak3/Tyk2/STAT1/STAT2/STAT3/STAT4/STAT5 a/STAT5b/STAT6/OSM/gp130/LIFR/OSM-Rβ.

In some embodiments, the JAK inhibitor is ruxolitinib (INCB 018424), tofacitinib (CP690550), Tyrphostin AG490 (CAS Number: 133550-30-8), momelotinib (CYT387), pacritinib (SB1518), baricitinib (LY3009104), fedratinib (TG101348), BMS-911543 (CAS Number: 1271022-90-2), lestaurtinib (CEP-701), fludarabine, epigallocatechin-3-gallate (EGCG), peficitinib, ABT 494 (CAS Number: 1310726-60-3), AT 9283 (CAS Number: 896466-04-9), decernmotinib, filgotinib, gandotinib, INCB 39110 (CAS Number: 1334298-90-6), PF 04965842 (CAS Number: 1622902-68-4), R348 (R-932348, CAS Number: 916742-11-5; 1620142-65-5), AZD 1480 (CAS Number: 935666-88-9), cerdulatinib, INCB 052793 (Incyte, clinical trial ID: NCT02265510), NS 018 (CAS Number: 1239358-86-1 (free base); 1239358-85-0 (HCl)), AC 410 (CAS Number: 1361415-84-0 (free base); 1361415-86-2 (HCl).), CT 1578 (SB 1578, CAS Number: 937273-04-6), JTE 052 (Japan Tobacco Inc.), PF 6263276 (Pfizer), R 548 (Rigel), TG 02 (SB 1317, CAS Number: 937270-47-8), lumbricus rebellus extract, ARN 4079 (Arrien Pharmaceuticals, LLC.), AR 13154 (Aerie Pharmaceuticals Inc.), UR 67767 (Palau Pharma S.A.), CS510 (Shenzhen Chipscreen Biosciences Ltd.), VR588 (Vectura Group plc), DNX 04042 (Dynamix Pharmaceuticals/Clevexel), hyperforin, or combinations thereof.

In some embodiments, the individual has had an inadequate response with, lost response to, been intolerant to, or demonstrated dependence on another agent for the treatment of AA. In some embodiments, the individual has had an inadequate response with the other agent for the treatment of AA. In some embodiments, the individual has lost response to another agent for the treatment of AA. In some embodiments, the individual was intolerant to another agent for the treatment of AA.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the individual has been intolerant to conventional therapy. In some embodiments, the prior conventional therapy is referred to as prior treatment.

In some embodiments, the individual had an inadequate response with, lost response to, or was intolerant to the at least one therapeutic agent or therapy. In some embodiments, the individual has demonstrated an inadequate response to, loss of response to, or intolerance of to the at least one therapeutic agent or therapy. In some embodiments, the individual had demonstrated, over the previous 3-month (12-week) period, an inadequate response to, loss of response to, or intolerance of the at least one therapeutic agent or therapy. In some embodiments, the individual had demonstrated, over the previous 6-month period, an inadequate response to, loss of response to, or intolerance of the at least one therapeutic agent or therapy. In some embodiments, the individual had demonstrated, over the previous 9-month period, an inadequate response to, loss of response to, or intolerance of the at least one therapeutic agent or therapy. In some embodiments, the individual had demonstrated, over the previous 1-year period, an inadequate response to, loss of response to, or intolerance of the at least one therapeutic agent or therapy. In some embodiments, the individual had demonstrated, over the previous 2-year period, an inadequate response to, loss of response to, or intolerance of the at least one therapeutic agent or therapy. In some embodiments, the individual had demonstrated, over the previous 3-year period, an inadequate response to, loss of response to, or intolerance of the at least one therapeutic agent or therapy. In some embodiments, the individual had demonstrated, over the previous 4-year period, an inadequate response to, loss of response to, or intolerance of the at least one therapeutic agent or therapy. In some embodiments, the individual had demonstrated, over the previous 5-year period, an inadequate response to, loss of response to, or intolerance of the at least one therapeutic agent or therapy.

In some embodiments, the individual does not have androgenetic alopecia, cicatricial (scarring) alopecia, secondary syphilis, Tinea capitis, trichotillomania, or triangular alopecia. In some embodiments, the individual does not have cicatricial (scarring) alopecia. In some embodiments, the individual does not have central centrifugal cicatricial alopecia. In some embodiments, the individual does not have traction alopecia. In some embodiments, the individual does not have androgenetic alopecia.

In some embodiments, the method further comprises monitoring for adverse events during the administration of Compound 1, or a pharmaceutically acceptable salt, hydrate, or solvate thereof, and optionally, interrupting or terminating the administration of Compound 1, or a pharmaceutically acceptable salt, hydrate, or solvate thereof.

In some embodiments, the treatment further comprises monitoring heart rate during the administration, monitoring pulmonary function during the administration, or monitoring liver function during the administration.

In some embodiments, the treatment further comprises monitoring heart rate during the administration.

In some embodiments, the treatment further comprises monitoring pulmonary function during the administration.

In some embodiments, the treatment further comprises monitoring liver function during the administration.

In some embodiments, the method reduces the incidence and severity of adverse events resulting from the treatment of a condition described herein.

In some embodiments, the adverse event is a serious adverse event.

In some embodiments, the serious adverse event is selected from leukopenia, constipation, diarrhea, nausea, abdominal pain, neutropenia, vomiting, back pain, and menstrual disorder.

In some embodiments, the method results in no serious adverse events.

In some embodiments, the standard dose is administered without substantially inducing an acute heart rate reduction or heart block in the individual.

In some embodiments, Compound 1, or a pharmaceutically acceptable salt, hydrate, or solvate thereof, is administered without causing a reduction of more than 6 bpm in heart rate.

In some embodiments, Compound 1, or a pharmaceutically acceptable salt, hydrate, or solvate thereof, is administered without a first-dose effect on heart rate as seen with other S1P receptor modulators. In some embodiments, Compound 1, or a pharmaceutically acceptable salt, hydrate, or solvate thereof, is administered without a first-dose effect on AV conduction as seen with other S1P receptor modulators.

In some embodiments, the treatment is for inducing clinical remission. In some embodiments, the treatment is for maintaining clinical remission. In some embodiments, the treatment is for inducing and maintaining clinical remission.

In some embodiments, treating is reducing a sign and/or symptom of alopecia areata, such as reducing hair loss. In some embodiments, treating is reducing a sign of alopecia areata. In some embodiments, treating is reducing a symptom of alopecia areata.

In some embodiments, treating is inducing and/or maintaining clinical remission, such as inducing and/or maintaining hair growth. In some embodiments, treating is inducing and maintaining clinical remission.

In some embodiments, treating is inducing and/or maintaining clinical response, such as inducing and/or maintaining hair growth. In some embodiments, treating is inducing and maintaining clinical response. In some embodiments, treating is inducing clinical response. In some embodiments, treating is maintaining clinical response.

In some embodiments, treating is reducing signs and symptoms and inducing and maintaining clinical remission of alopecia areata in an individual who has had inadequate response to conventional therapy. In some embodiments, treating is reducing signs and symptoms and inducing and maintaining clinical remission of alopecia areata in an individual who has lost response to or is intolerant to a conventional therapy. In some embodiments, treating is reducing signs and symptoms and inducing and maintaining clinical response in an individual with alopecia areata who has had inadequate response to conventional therapy. In some embodiments, treating is reducing signs and symptoms and inducing and maintaining clinical response in an individual with alopecia areata who has lost response to or is intolerant to a conventional therapy.

In some embodiments, Compound 1, or a pharmaceutically acceptable salt, hydrate, or solvate thereof, is administered without causing a severe adverse event. In some embodiments, Compound 1, or a pharmaceutically acceptable salt, hydrate, or solvate thereof, is administered without causing a severe adverse event related to heart rate. In some embodiments, Compound 1, or a pharmaceutically acceptable salt, hydrate, or solvate thereof, is administered without causing a severe adverse event related to heart rate change. In some embodiments, Compound 1, or a pharmaceutically acceptable salt, hydrate, or solvate thereof, is administered without causing a severe adverse event related to elevated heart rate. In some embodiments, Compound 1, or a pharmaceutically acceptable salt, hydrate, or solvate thereof, is administered without causing a severe adverse event related to bradycardia. In some embodiments, Compound 1, or a pharmaceutically acceptable salt, hydrate, or solvate thereof, is administered without causing a severe adverse event related to AV block. In some embodiments, Compound 1, or a pharmaceutically acceptable salt, hydrate, or solvate thereof, is administered without causing a severe adverse event related to AV conduction. In some embodiments, Compound 1, or a pharmaceutically acceptable salt, hydrate, or solvate thereof, is administered without causing bradycardia. In some embodiments, Compound 1, or a pharmaceutically acceptable salt, hydrate, or solvate thereof, is administered without causing AV block. In some embodiments, Compound 1, or a pharmaceutically acceptable salt, hydrate, or solvate thereof, is administered without causing more than mild decrease in heart rate on first day of treatment (for example, >10 bpm). In some embodiments, Compound 1, or a pharmaceutically acceptable salt, hydrate, or solvate thereof, is administered without a first-dose effect seen with other S1P receptor modulators. In some embodiments, Compound 1, or a pharmaceutically acceptable salt, hydrate, or solvate thereof, is administered without a first-dose cardiovascular effect seen with other S1P receptor modulators. In some embodiments, Compound 1, or a pharmaceutically acceptable salt, hydrate, or solvate thereof, is administered without symptomatic changes in heart rate. In some embodiments, Compound 1, or a pharmaceutically acceptable salt, hydrate, or solvate thereof, is administered without symptomatic changes in heart rhythm. In some embodiments, Compound 1, or a pharmaceutically acceptable salt, hydrate, or solvate thereof, is administered without requiring titration to avoid first-dose effect seen with other S1P receptor modulators.

In some embodiments, Compound 1, or a pharmaceutically acceptable salt, hydrate, or solvate thereof, is administered without increasing a liver function test (LFT). In some embodiments, Compound 1, or a pharmaceutically acceptable salt, hydrate, or solvate thereof, is administered without causing an elevated LFT. In some embodiments, Compound 1, or a pharmaceutically acceptable salt, hydrate, or solvate thereof, is administered without increasing ALT. In some embodiments, Compound 1, or a pharmaceutically acceptable salt, hydrate, or solvate thereof, is administered without increasing AST. In some embodiments, Compound 1, or a pharmaceutically acceptable salt, hydrate, or solvate thereof, is administered without increasing ALT>3X ULN. In some embodiments, Compound 1, or a pharmaceutically acceptable salt, hydrate, or solvate thereof, is administered without increasing ALT>2.5X ULN. In some embodiments, Compound 1, or a pharmaceutically acceptable salt, hydrate, or solvate thereof, is administered without increasing ALT>2X ULN. In some embodiments, Compound 1, or a pharmaceutically acceptable salt, hydrate, or solvate thereof, is administered without increasing ALT>1.5X ULN. In some embodiments, Compound 1, or a pharmaceutically acceptable salt, hydrate, or solvate thereof, is administered without increasing AST>3X ULN. In some embodiments, Compound 1, or a pharmaceutically acceptable salt, hydrate, or solvate thereof, is administered without increasing AST>2.5X ULN. In some embodiments, Compound 1, or a pharmaceutically acceptable salt, hydrate, or solvate thereof, is administered without increasing AST>2X ULN. In some embodiments, Compound 1, or a pharmaceutically acceptable salt, hydrate, or solvate thereof, is administered without increasing AST>1.5X ULN. In some embodiments, Compound 1, or a pharmaceutically acceptable salt, hydrate, or solvate thereof, is administered without increasing bilirubin. In some embodiments, Compound 1, or a pharmaceutically acceptable salt, hydrate, or solvate thereof, is administered without increasing bilirubin>3X ULN. In some embodiments, Compound 1, or a pharmaceutically acceptable salt, hydrate, or solvate thereof, is administered without increasing bilirubin>2.5X ULN. In some embodiments, Compound 1, or a pharmaceutically acceptable salt, hydrate, or solvate thereof, is administered without increasing bilirubin>2X ULN. In some embodiments, Compound 1, or a pharmaceutically acceptable salt, hydrate, or solvate thereof, is administered without increasing bilirubin>1.5X ULN. In some embodiments, Compound 1, or a pharmaceutically acceptable salt, hydrate, or solvate thereof, is administered without increasing gamma-glutamyl transferase (GGT). In some embodiments, Compound 1, or a pharmaceutically acceptable salt, hydrate, or solvate thereof, is administered without increasing GGT>3X ULN. In some embodiments, Compound 1, or a pharmaceutically acceptable salt, hydrate, or solvate thereof, is administered without increasing GGT>2.5X ULN. In some embodiments, Compound 1, or a pharmaceutically acceptable salt, hydrate, or solvate thereof, is administered without increasing GGT>2X ULN. In some embodiments, Compound 1, or a pharmaceutically acceptable salt, hydrate, or solvate thereof, is administered without increasing GGT>1.5X ULN.

In some embodiments, Compound 1, or a pharmaceutically acceptable salt, hydrate, or solvate thereof, is administered without causing an abnormality in a pulmonary function test. In some embodiments, Compound 1, or a pharmaceutically acceptable salt, hydrate, or solvate thereof, is administered without causing macular edema.

In some embodiments, the Compound 1, or a pharmaceutically acceptable salt, hydrate, or solvate thereof, is administered orally.

In some embodiments, the Compound 1, or a pharmaceutically acceptable salt, hydrate, or solvate thereof, is formulated as a capsule or tablet suitable for oral administration.

In some embodiments, the Compound 1, or a pharmaceutically acceptable salt, hydrate, or solvate thereof, is selected from: Compound 1; a calcium salt of Compound 1; and an L-arginine salt of Compound 1. In some embodiments, the Compound 1, or a pharmaceutically acceptable salt, hydrate, or solvate thereof, is an L-arginine salt of Compound 1. In some embodiments, the Compound 1, or a pharmaceutically acceptable salt, hydrate, or solvate thereof, is an anhydrous, non-solvated crystalline form of an L-arginine salt of Compound 1. In some embodiments, the Compound 1, or a pharmaceutically acceptable salt, hydrate, or solvate thereof, is an anhydrous, non-solvated crystalline form of Compound 1.

Also provided are pharmaceutical compositions comprising a standard dose of Compound 1, or, a pharmaceutically acceptable salt, a hydrate or solvate thereof and, optionally, one or more pharmaceutically acceptable carriers. Also provided are pharmaceutical compositions comprising Compound 1, or, a pharmaceutically acceptable salt, a hydrate or solvate thereof, optionally, one or more pharmaceutically acceptable carriers. The carrier(s) must be “acceptable” in the sense of being compatible with the other ingredients of the formulation and not overly deleterious to the recipient thereof

In some embodiments, Compound 1, or, a pharmaceutically acceptable salt, a hydrate or solvate thereof, is administered as a raw or pure chemical, for example as a powder in capsule formulation.

In some embodiments, Compound 1, or, a pharmaceutically acceptable salt, a hydrate or solvate thereof, is formulated as a pharmaceutical composition further comprising one or more pharmaceutically acceptable carriers. In some embodiments, Compound 1, or a pharmaceutically acceptable salt, hydrate, or solvate thereof, is the sole active agent in the pharmaceutical compositions. In some embodiments, Compound 1, or a pharmaceutically acceptable salt, hydrate, or solvate thereof, is the sole active ingredient in the pharmaceutical dosage form.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20th Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules, or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary. Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1

Formulations composed of immediate-release, hard gelatin capsules containing an L-arginine salt of Compound 1 were prepared as shown in Table 1.

TABLE 1

| | Formulation | | | | |
|---|---|---|---|---|---|
| | 0.1 mg | 0.35 mg | 0.5 mg | 1 mg | 2 mg |
| L-arginine salt of | 0.14 | 0.48 | 0.69 | 1.38 | 2.76 |
| Compound 1 (mg/capsule) | | | | | |
| Empty capsule weight (mg)* | 38.0 | 61.0 | 61.0 | 61.0 | 61.0 |
| Total capsule target | 38.14 | 61.48 | 61.69 | 62.38 | 63.76 |
| weight (mg)** | | | | | |

*Approximate weight. Based on capsule specification
**Theoretical total weight calculated by combining fill and empty capsule weights together

### Example 2

Formulations composed of immediate-release tablets containing an L-arginine salt of Compound 1 were prepared as shown in Table 2.

TABLE 2

| Tablet Strength | 0.5 mg | 1 mg | 2 mg | 3 mg |
|---|---|---|---|---|
| L-Arg Salt of Compound 1 | 0.69 | 1.381 | 2.762 | 4.143 |
| Mannitol Pearlitol ® 100SD | 54.81 | 54.119 | 52.738 | 51.357 |
| Microcrystalline cellulose - | 40 | 40 | 40 | 40 |
| Sodium Starch Glycolate - | 4 | 4 | 4 | 4 |
| Magnesium Stearate | 0.5 | 0.5 | 0.5 | 0.5 |
| Opadry ® II Blue | 4 | 4 | 4 | 4 |
| Total tablet target weight | 104 | 104 | 104 | 104 |

### Example 3

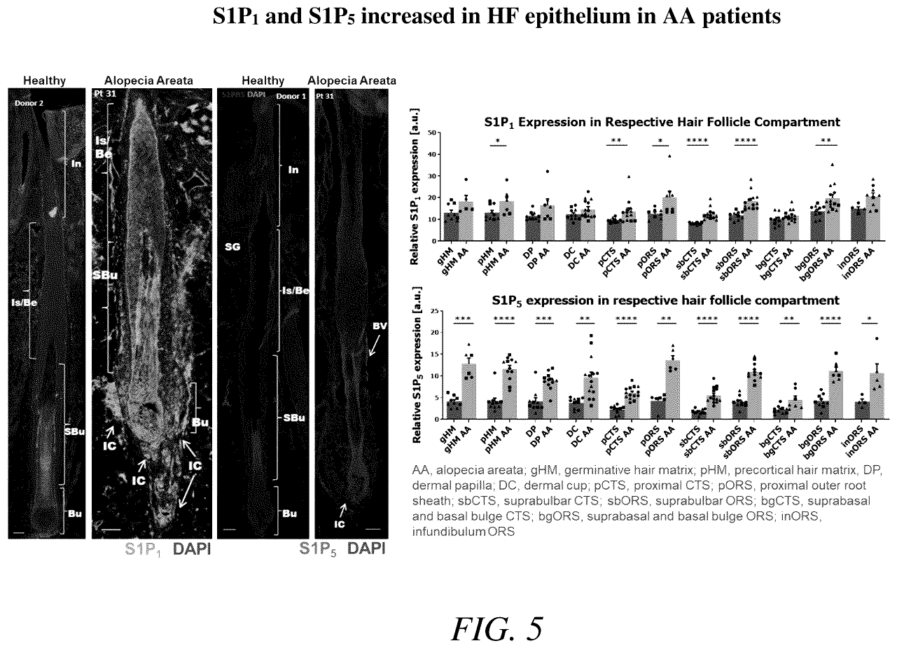

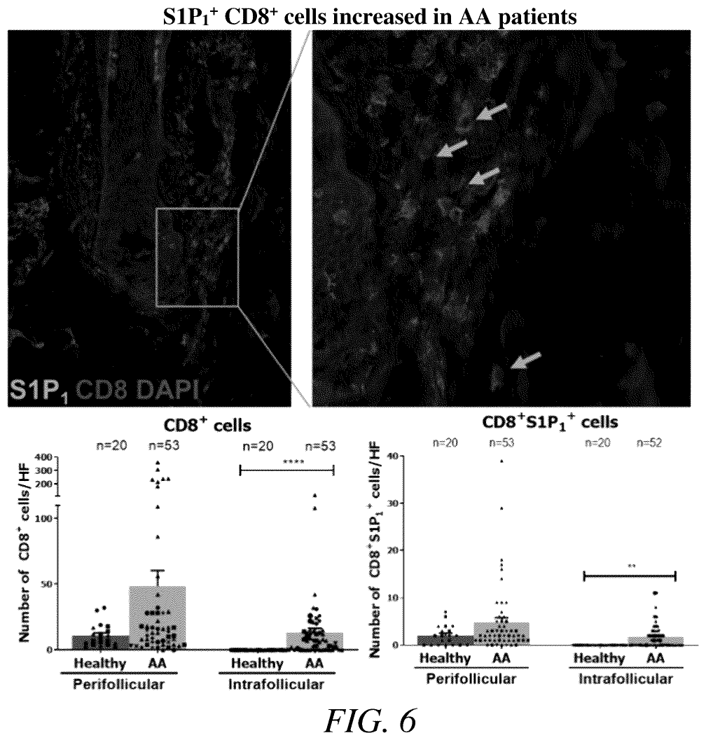

In the skin, S1P receptor signaling is known to be involved in epidermal keratinocyte proliferation and differentiation (Vogler 2003). Given that scalp hair follicles also harbor keratinocytes, and are the target of autoimmune responses, Compound 1 (etrasimod) may be beneficial for hair follicle (HF) physiology and pathology. By RNAseq, in situ hybridization, and immunofluorescence, it was confirmed that, similar to skin keratinocytes, S1P₁ and S1P₅ are expressed by the HF epithelium and mesenchyme in scalp skin. Freshly frozen scalp biopsies from 3 healthy donors and 3 AA patients were immunostained for S1P₁ and S1P₅, and expression was quantified in distinct regions of the hair follicle and immune cell infiltrate. As shown in FIG. 5, compared to healthy controls, the intrafollicular epithelial expression of S1P₁ and S1P₅ was found to be increased in patients suffering from alopecia areata (AA). As shown in FIG. 6, AA HFs also exhibited a perifollicular and intrafollicular immune cell infiltrate, which included CD8⁺ T cells, that is characteristic of this disease. Within this infiltrate, AA HFs showed an increase of S1P₁⁺ immune cells, including CD8+ cells. Together, this data shows that the S1P receptors that are modulated by Compound 1 are increased in both the hair follicle and immune cell infiltrate in AA patients, suggesting potential to be impacted by Compound 1.

A hair follicle organ culture model was used to analyze the direct effects of Compound 1 on HF physiology in a non-inflammatory condition. Human full thickness scalp skin from four healthy donors was cultured ex vivo and treated in four groups with 11 HF's per groups: 1. vehicle (DMSO control), 2. 10 nM Compound 1, 3. 100 nM Compound 1, 4. 1 μM Compound 1. Before treatment began, 54 full length microdissected HF's were isolated, pictures and length measurements were taken of the HF's, and the HF's were cultured in WCM. On Days 1, 3, and 5 pictures of HF elongation were taken, the medium was changed, and treatment in groups 1-4 occurred. Days 2 and 4 were rest days. Additionally, on Day 2 three HF's per group were frozen for RNAseq twenty-four hours after the previous day's treatment. On Day 6, pictures were taken of the HF elongation, the culture was closed, and 8 HF's per group were embedded in optimal cutting temperature compound (OCT compound) for further microscopic immunohistological analysis.

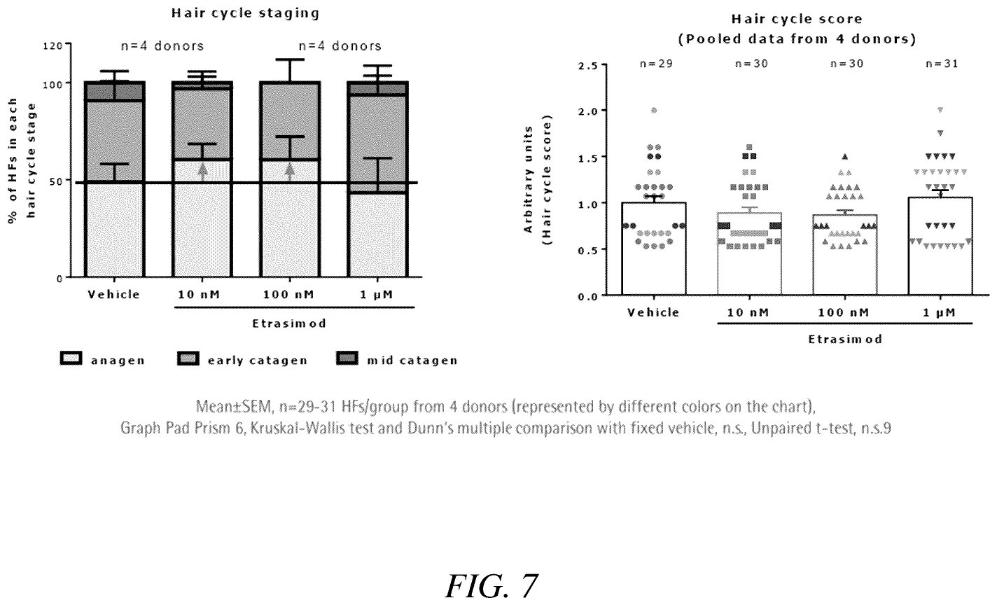

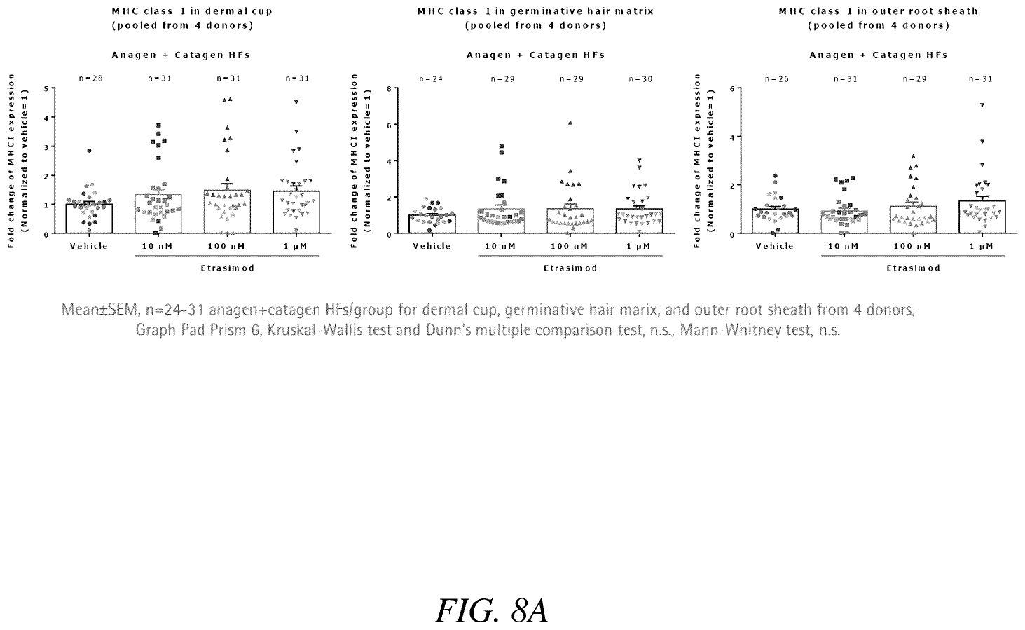

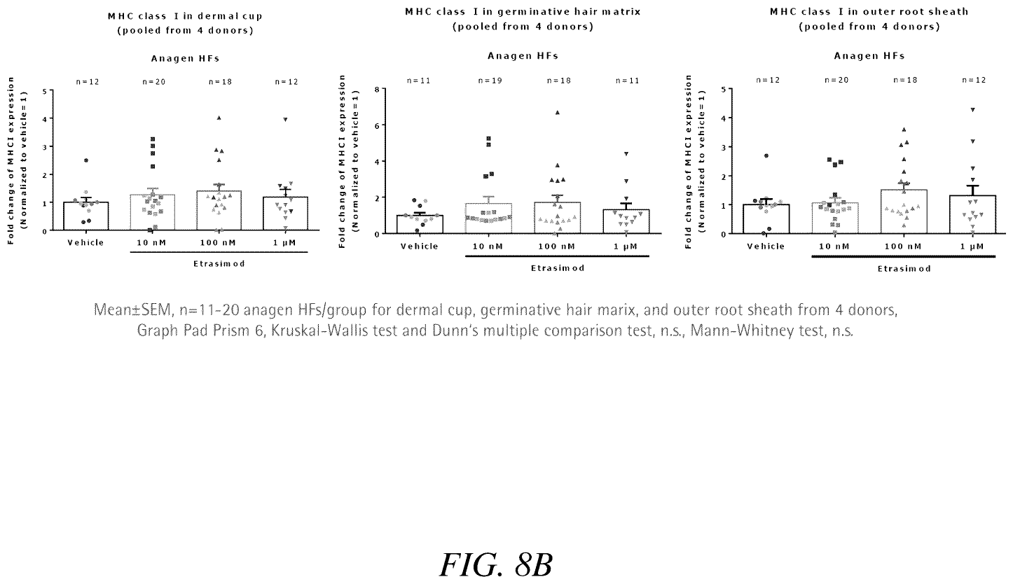

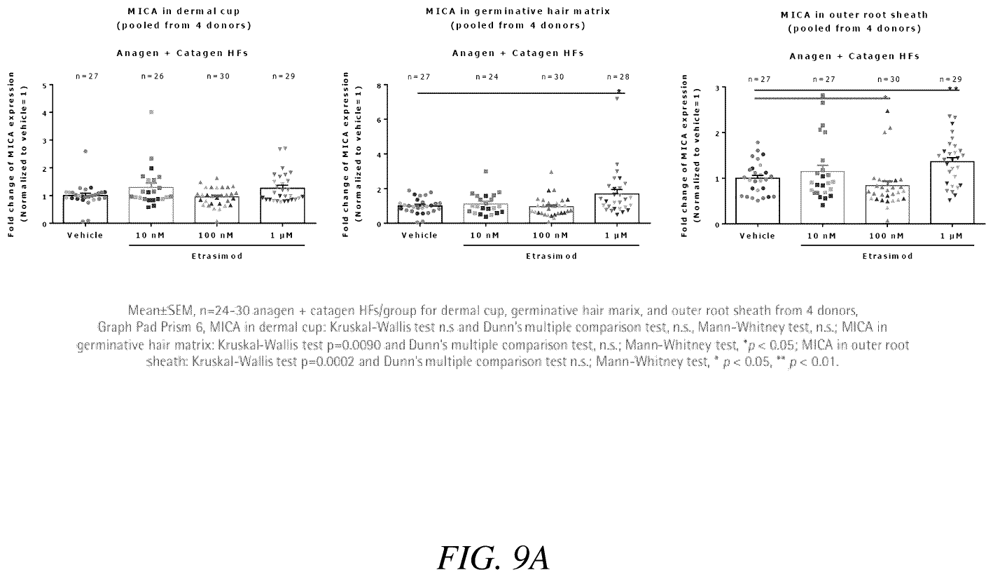

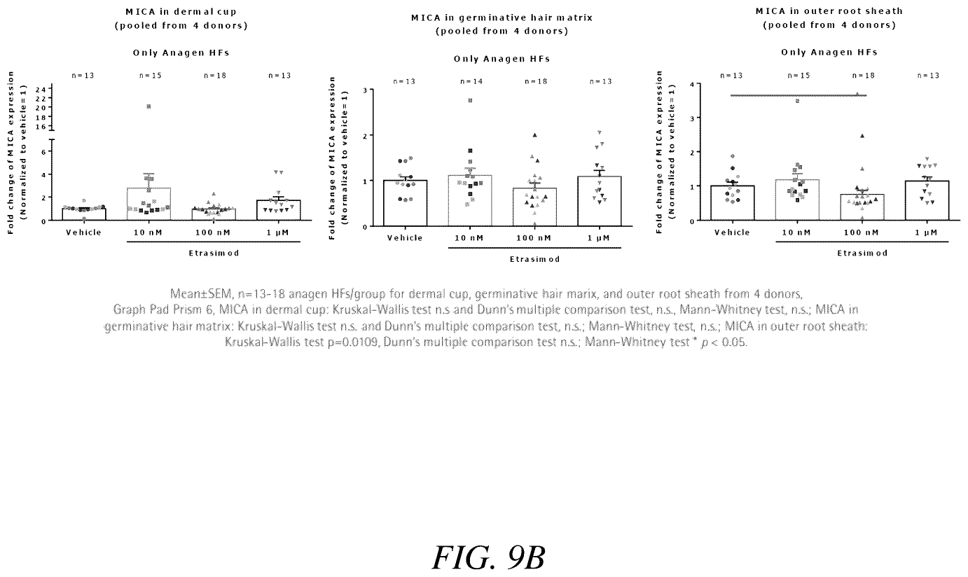

As shown in FIG. 7, 10 nM and 100 nM of Compound 1 treatment tendentially prolonged the anagen phase of the treated HF's. This suggests a potentially beneficial role of the drug in extending the growth phase and preventing hair shedding. As shown in FIGS. 8a and 8b, no change in Major Histocompatibility Complex (MHC) Class I (MHC-I) expression was observed in anagen and catagen HFs combined and anagen hair follicles alone. This demonstrates the preservation of the HF's immune privilege, which is relevant in AA patients in which the hair follicles lose their immune privilege. As shown in FIG. 9, 100 nM of Compound 1 significantly reduced MICA, a stress signal that is increased in AA, in anagen and catagen HFs combined and anagen hair follicles alone. RNAseq data revealed a significant up-regulation of genes involved in hair keratinization in the 10 nm and 100 nm groups. Altogether this data suggests that Compound 1 has impacts on hair follicle physiology and has potential therapeutic utility for the treatment of AA.

To evaluate the effects of Compound 1 on hair follicle physiology and pathology in an AA-like inflammatory environment, a human full thickness scalp skin model was used. Scalp biopsies from two healthy donors were cultured ex vivo and treated with tofacitinib (400 nM) and Compound 1 (10 nM, 100 nM, 1000 nM) in the presence and absence of IFNγ, a key pathological cytokine involved in AA development. 5 mm skin cubes with hair were acquired from a 56 year old female donor (Donor 1) and a 44 year old female donor (Donor 2). Ten groups were tested with one skin punch per group: 1. Vehicle (DMSO control) 2. Tofacitinib (400 nM), 3. 10 nM Compound 1, 4. 100 nM Compound 1 5. 1 μM Compound 1, 6. Vehicle (DMSO control)+100 IU IFNγ, 7. Tofacitinib (400 nM)+100 IU IFNγ, 8. 10 nM Compound 1+100 IU IFNγ, 9. 100 nM Compound 1+100 IU IFNγ 10. 1 μM Compound 1+100 IU IFNγ. On Day 0, isolation of 10×6mm punches with hair was performed. On Days 1, 3, and 5 the medium was changed and treatment with groups 1-10 occurred. Days 2 and 4 were rest days. On Day 6, the culture was closed and embedded in OCT Compound for further microscopic analysis.

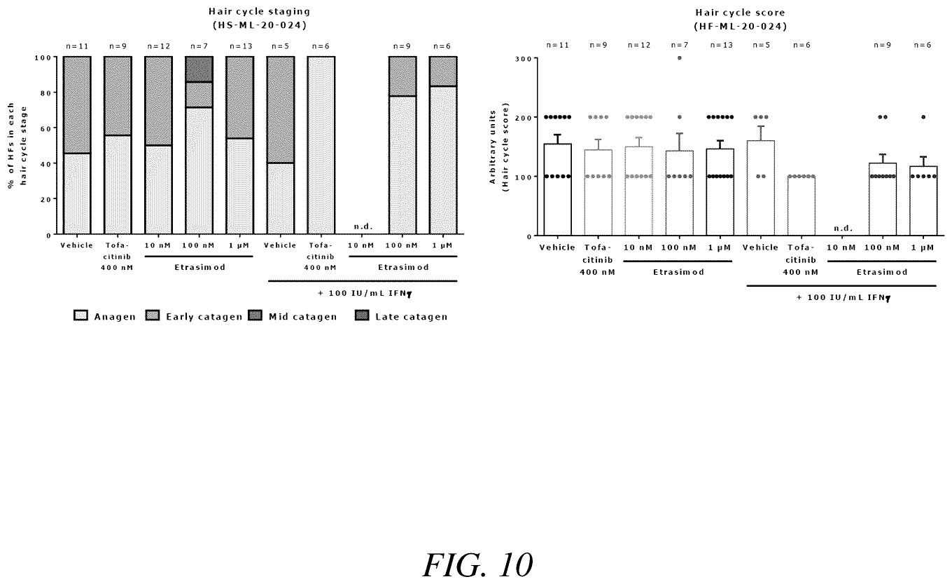

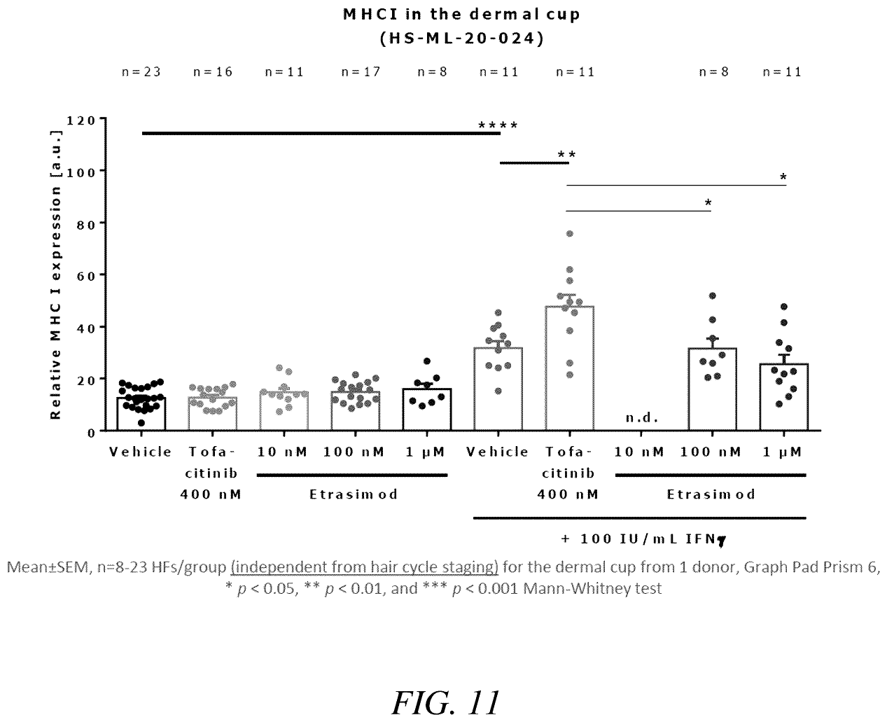

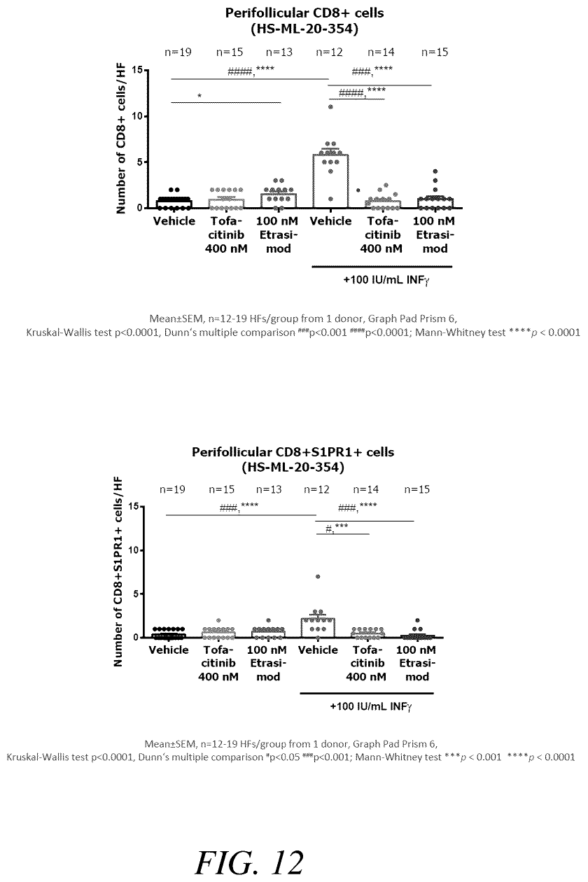

As shown in FIG. 10, in Donor 1 Compound 1 (etrasimod) tendentially prolonged anagen, even more so in presence of IFNγ. As shown in FIG. 11, the administration of Compound 1 prevented IFNγ-mediated MHC-I up-regulation in the dermal cup of the hair follicle. As shown in FIG. 12, in Donor 2 the increase of CD8+ and CD8-S1P₁+ cells showing lymphocytic morphology was prevented by Compound 1. These results suggest that Compound 1 may assist in the treatment of AA by prolonging the anagen phase and preventing experimentally-induced AA immune privilege collapse.

### Example 4

A phase 2, double-blind, placebo controlled clinical trial will be conducted in individuals with moderate to severe alopecia areata. The trial will evaluate once daily oral administration of Compound 1 (2 mg) or placebo for up to 52 weeks. The study will include a screening period, a 24 week double-blind treatment period, a 28 week open-label extension period, and a safety follow-up period. Subjects will be randomized in a 2:1 ratio to receive Compound 1 or placebo in a double-blinded manner. Approximately thirty-six subjects with moderate-to-severe AA with a current episode of hair loss for ≥6 months but <8 years will be randomized in a 2:1 ratio to receive a Compound 1 (2 mg) tablet or placebo tablet orally, once daily, in a double-blind manner for twenty-four weeks. Approximately six subjects who have alopecia totalis or alopecia universalis will be recruited to participate in the study. Randomization is stratified by SALT I score (<100, 100) at Day 1/Baseline. During the open-label extension period, all eligible subjects will receive 2 mg of Compound 1 orally, once daily.

Subjects ≥18 and ≤70 years of age with alopecia areata affecting at least 50% of the scalp area (SALT score of at least 50) will be eligible. Subjects will have stable or worsening disease for at least 6 months and less than 8 years. Approximately thirty-six subjects are planned to be enrolled in the study, including approximately twenty-four subjects in the 2 mg Compound 1 group and twelve subjects in the placebo group during the double-blind treatment period. The target is to enroll approximately six subjects who have alopecia totalis or alopecia universalis.

### Inclusion Criteria:

Subjects must meet ALL of the following inclusion criteria to be eligible for enrollment into the study:

Key inclusion criteria
1. Men or women between ≥18 and ≤70 years of age at the time of informed consent
2. Moderate-to-severe AA as assessed by a SALT I score of ≥50
3. Current episode of hair loss for ≥6 months but <8 years
4. Stable disease condition (i.e., no significant growth or loss of hair) in the last 6 months as assessed by the Investigator
5. Willing to keep the same hair style and color (e.g., hair products, process, and timing for hair appointments) for the duration of the study:
   Key exclusionary criteria
1. History of male or female pattern hair loss >Hamilton stage III or >Ludwig stage II
2. Other types of alopecia (e.g., cicatricial/scarring alopecia [including central centrifugal cicatricial alopecia], traction alopecia, or telogen effluvium) or other diseases that could cause hair loss
3. Active scalp inflammation, scalp infection, scalp psoriasis, or any other scalp condition that may interfere with the SALT I assessment
4. Previous use of an oral JAK inhibitor, including participation in clinical studies of JAK inhibitors
   Exclusionary criteria related to medications, therapies, or skin disease
5. Phototherapy on scalp within 4 weeks of screening
6. Treatment with the following medications within 12 weeks of screening:
   a. Topical, intralesional, or systemic corticosteroids
   b. Topical immunotherapy (e.g., diphenylcyclopropenone, squaric acid)
   c. Topical calcineurin inhibitors
   d. Topical JAK inhibitors
   e. Topical or oral minoxidil
   f. Systemic glucocorticoids (excludes inhaled or intranasal delivery that are considered topical for any reason)
   g. Immunoglobulin or blood products
7. Use of any biological agents (e.g., dupilumab, adalimumab, ustekinumab, secukinumab) regardless of indication or systemic immunosuppressive/immunomodulating drugs (e.g., cyclosporine, azathioprine, methotrexate) within 5 half-lives (if known) or 12 weeks before screening, whichever is longer
8. Use of sphingosine 1-phosphate receptor modulators (e.g., fingolimod, siponimod, ozanimod), α4β1-integrin receptor antagonists (e.g., natalizumab), and lymphocyte-depleting therapies (e.g., rituximab, cyclophosphamide, bone marrow transplantation, total body irradiation) within 6 months before screening or until lymphocyte count returns to normal, whichever is longer
9. History of or planned hair transplant procedure during the study
10. Planned microblading or micropigmentation of the scalp during the study
11. Received any investigational agent, including non-biologic agents and topical agents, within 5 half-lives (if known) or 4 weeks (whichever is longer) before screening
12. Moderate or strong inducers/inhibitors of cytochrome P450 (CYP) 2C8 or CYP2C9 (e.g., clopidogrel, gemfibrozil, fluconazole, carbamazepine, and St. John's Wort), or uridine diphosphate (UDP) glucuronosyltransferase (UGT) family 1 member A7 (UGT1A7) use within 4 weeks before screening
   Exclusionary criteria related to medical history
13. Known active bacterial, viral, fungal, mycobacterial infection, or other infection (including tuberculosis [TB] or atypical mycobacterial disease) or any major episode of infection that required hospitalization or treatment with intravenous antibiotics within 4 weeks before screening or during screening, or oral antibiotics within 2 weeks before screening or during screening. Superficial fungal infection of the nail bed is allowed
14. Have any of the following conditions or risk factors:
a. Primary or secondary immunodeficiency syndromes (e.g., hereditary immunodeficiency syndrome, acquired immunodeficiency syndrome, drug-induced immune deficiency)
b. History of organ transplant (except corneal transplant)
c. History of an opportunistic infection (e.g., Pneumocystis jirovecii pneumonia, cryptococcal meningitis, progressive multifocal leukoencephalopathy [PML])
d. History of disseminated herpes simplex or disseminated herpes zoster, or any episode of herpes zoster
e. Test positive for human immunodeficiency virus, hepatitis B virus (positive for hepatitis B surface antigen [HBsAg]), or active hepatitis C virus (HCV) (positive HCV antibody with detectable viral load) at screening
f. History of active or latent TB
15. Received any live or live-attenuated vaccines within four weeks before screening
16. History of malignancy of any organ system (other than localized squamous cell or basal cell carcinoma of the skin that have been excised or resolved), treated or untreated, within the past 5 years
17. Have any of the following conditions or receiving treatments that may affect cardiovascular function:
a. Myocardial infarction, unstable angina, stroke/transient ischemic attack, decompensated heart failure requiring hospitalization or Class III/IV heart failure within 8 weeks before screening
b. Second-degree or third-degree atrioventricular block, sick sinus syndrome without a functional pacemaker, or periods of asystole for >3 seconds without an implanted cardiac defibrillator
c. Recurrent symptomatic bradycardia or recurrent cardiogenic syncope
d. screening or Day 1 pre-randomization vital signs (taken in the sitting position) with a heart rate (HR)<50 beats per minute (bpm), systolic blood pressure (BP)<90 mm Hg, OR diastolic BP<55 mm Hg. Vital signs may be repeated up to 3 times during a visit to confirm abnormal readings
e. Screening or Day 1 pre-randomization electrocardiogram (ECG) with PR interval≥200 ms or QT interval corrected using Fridericia's formula (QTcF)≥450 milliseconds (ms) in males or ≥470 ms in females
f. Start, stop, or change dosage of Class I-IV anti-arrhythmic drugs within 1 week of screening
18. A history of or active diabetic retinopathy, uveitis, retinitis pigmentosum, or macular edema. Any recent intraocular surgery within 1 year of screening
19. Active severe pulmonary disease (e.g., chronic obstructive pulmonary disease or pulmonary fibrosis) or chronic pulmonary disease requiring intravenous corticosteroid treatment or hospitalization within 12 months before screening or during the screening period
20. Have forced expiratory volume at 1 second (FEV1) or forced vital capacity (FVC)<70% of predicted values at screening
21. Have any uncontrolled systemic disease(s) (e.g., thyroid disorder, hypertension, diabetes). If the condition is considered controlled and the subject is on any medications (e.g., thyroid medication or hormonal therapy) for treatment of diseases, the subject may be allowed to participate but must have been on a stable dose for at least 6 months prior to screening and remain on a stable dose throughout the study.
Exclusionary criteria related to test or laboratory results (performed by central laboratory) Note: A confirmed result means there have been 2 consecutive assessments showing a consistent abnormal, clinically relevant result.
22. Confirmed absolute lymphocyte count<0.8×109 cells/L at screening
23. Confirmed estimated glomerular filtration rate<30 mL/min/1.73 m2 by the Chronic Kidney Disease Epidemiology Collaboration equation at screening
24. Confirmed aspartate aminotransferase (AST) or alanine aminotransferase (ALT)>2×upper limit of normal (ULN) and total bilirubin>1.5×ULN (unless consistent with a history of Gilbert's Syndrome) at screening
   General exclusionary criteria
25. Lactating female who is breastfeeding
26. Any acute illness or medical condition including psychiatric disease, cognitive impairment, and alcohol/drug abuse/dependence, or signs/symptoms suspicious for a serious disease that, in the Investigator's opinion, could put the subject at increased risk for safety event(s), could interfere with participation in the study according to the study protocol, or with the ability of the subject to cooperate and comply with the study procedures

### Exclusion Criterion (for the Open-Label Extension Period):

Subjects will be excluded from the Open-Label Extension Period if they meet the following exclusion criterion at the Week 24 visit:
1. Week 24 predose vital signs (taken in the sitting position) with an HR<50 bpm, systolic BP<90 mm Hg, or diastolic BP<55 mm Hg. Vital signs may be repeated up to 3 times during the visit to confirm abnormal readings

The severity of alopecia areata and clinical response will be measured as detailed in the Alopecia Areata Investigational Guidelines. Hair regrowth will be reflected by a decrease in the SALT score (e.g., complete hair regrowth would confer a SALT score of 0). Efficacy endpoints will include the percentage of participants with at least a 30%, 50%, 75%, and 90% improvement from the baseline in their SALT score at week 24. The SALT score will be measured visually by the study physician and corroborated by photographic analysis. The SALT 1 score will be assessed at screening, day 1, week 2, week 4, week 8, week 12, week 20, and week 24 of the 24-week treatment period, and week 28, week 26, week 44, and week 52 of the open-label extension period.

Efficacy assessments include percent change, change, and categorical percent change in hair loss (SALT I score); the following patient-reported outcomes: AA Symptom Impact Scale (AASIS) and AA Quality of Life Index (AA-QLI); serum biomarkers; and photographs of the full scalp for all subjects, of the eyebrows and eyelashes for subjects who have hair loss in these areas at Day 1/Baseline, and of the fingernails for subjects with fingernail changes related to AA (e.g., pitting, white spots, and roughness) at Day 1/baseline. The definitions used to assess the primary, secondary, and exploratory efficacy outcomes are described below. The photographic assessments will not be statistically summarized as endpoints given the lack of formal scales and standardized measures of change for these evaluations.

### Primary Efficacy Endpoint

Percent change from baseline in SALT I at Week 24

### Secondary Efficacy Endpoints

Change from baseline in SALT I at Week 24
Proportion of subjects achieving a 30% improvement from baseline in SALT I (SALT30) at Week 24
Proportion of subjects achieving a 50% improvement from baseline in SALT I (SALT50) at Week 24
Proportion of subjects achieving a 75% improvement from baseline in SALT I (SALT75) at Week 24

### Exploratory Efficacy Endpoints

Percent change from baseline in SALT I over time
Change from Baseline in SALT I over time
Proportion of subjects achieving a 30% improvement from baseline in SALT I (SALT30) over time
Proportion of subjects achieving a 50% improvement from baseline in SALT I (SALT50) over time
Proportion of subjects achieving a 75% improvement from baseline in SALT I (SALT75) over time
Proportion of subjects achieving a 90% improvement from baseline in SALT I (SALT90) at Week 24
Change from baseline in Alopecia Areata Symptom Impact Scale (AASIS) at Week 24
Change from baseline in Alopecia Areata Quality of Life Questionnaire AA-QLI at Week 24
Change from baseline in serum biomarkers at Week 24
Percent change from baseline in peripheral lymphocyte counts at Week 24

Biomarker assessments with tissue biopsies and serum will be conducted. Cytokines and chemokines (e.g., interferon gamma [IFN-γ], interleukin [IL]-2, IL-12, IL-13, IL-10, and IL-17) may be measured by enzyme-linked immunosorbent assay, mass spectrometry, or comparable technology.

Other uses of the disclosed methods will become apparent to those in the art based upon, inter alia, a review of this patent document.

What is claimed is:

1. A method of treating alopecia areata (AA) in an individual in need thereof comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof.

2. The method of claim 1, further comprising detecting in the individual an AA-associated biomarker.

3. The method of claim 2, wherein the AA-associated biomarker is indicative of severity of alopecia areata.

4. The method of claim 2, wherein the AA-associated biomarker is indicative of the propensity of the individual to respond to treatment with Compound 1, or a pharmaceutically acceptable salt thereof.

5. The method of any one of the above claims, wherein prior to administering Compound 1, or a pharmaceutically acceptable salt thereof, to the individual, the method further comprises selecting the individual based on a level of an AA-associated biomarker.

6. The method of any one of the preceding claims, wherein the administration of Compound 1, or a pharmaceutically acceptable salt thereof, results in a change in a level of an AA-associated biomarker in the individual.

7. The method of any one of the preceding claims, wherein the individual has severe alopecia areata.

8. The method of any one of the preceding claims, wherein the individual has moderate alopecia areata.

9. The method of any one of claims 1 to 8, wherein the individual has diffuse alopecia areata.

10. The method of any one of claims 1 to 8, wherein the individual has alopecia areata monolocularis.

11. The method of any one of claims 1 to 8, wherein the individual has alopecia areata multilocularis.

12. The method of any one of claims 1 to 8, wherein the individual has ophiasis.

13. The method of any one of claims 1 to 8, wherein the individual has alopecia areata barbae.

14. The method of any one of claims 1 to 7, wherein the individual has alopecia areata totalis.

15. The method of any one of claims 1 to 7, wherein the individual has alopecia areata universalis.

16. The method of any one of the preceding claims, wherein the Compound 1, or a pharmaceutically acceptable salt thereof, is administered in combination with a second therapeutic agent or therapy.

17. The method of any one of the preceding claims, wherein the individual has demonstrated an inadequate response to, loss of response to, or intolerance of to at least one therapeutic agent or therapy.

18. The method of any one of the preceding claims, wherein the dosage form is administered under fasted conditions.

19. The method of any one of claims 1 to 17, wherein the dosage form is administered under fed conditions.

20. The method of any one of the preceding claims, wherein the therapeutically effective amount is equivalent to about 0.5 to about 5.0 mg of Compound 1.

21. The method of claim 20, wherein the therapeutically effective amount is in an amount equivalent to 2 mg of Compound 1.

22. The method of claim 20, wherein the individual is administered an amount equivalent to 2 mg of Compound 1 for a first time period and subsequently an amount equivalent to 3 mg of Compound 1 for a second time period.

23. The method of claim 20, wherein the therapeutically effective amount is in an amount equivalent to 3 mg of Compound 1.

24. The method of any one of the preceding claims, wherein the dosage form is administered without titration.

25. The method of any one of the preceding claims, wherein the Compound 1, or a pharmaceutically acceptable salt thereof, is administered orally.

26. The method of any one of the preceding claims, wherein the Compound 1, or a pharmaceutically acceptable salt thereof, is formulated as a capsule or tablet suitable for oral administration.

27. The method of any one of the preceding claims, wherein the Compound 1, or a pharmaceutically acceptable salt thereof, is selected from:
Compound 1;
a calcium salt of Compound 1; and
an L-arginine salt of Compound 1.

28. The method of claim 27, wherein the Compound 1, or a pharmaceutically acceptable salt thereof, is an L-arginine salt of Compound 1.

29. The method of claim 28, wherein the Compound 1, or a pharmaceutically acceptable salt thereof, is an anhydrous, non-solvated crystalline form of an L-arginine salt of Compound 1.

30. The method of claim 27, wherein the Compound 1, or a pharmaceutically acceptable salt thereof, is an anhydrous, non-solvated crystalline form of Compound 1.

31. The method of any one of the preceding claims, wherein the therapeutically effective amount of Compound 1, or a pharmaceutically acceptable salt thereof is administered once daily to the individual.

32. The method of any one of the preceding claims, wherein the method is non-gender specific.

33. The method of any one of the preceding claims, wherein the individual was previously administered at least one therapeutic agent or therapy.

34. The method of claim 33, wherein the individual had an inadequate response with, lost response to, or was intolerant to the at least one therapeutic agent or therapy.

35. The method of any one of the preceding claims, wherein treating comprises inducing and/or maintaining clinical response and/or inducing and/or maintaining clinical remission.

36. The method of any one of the preceding claims, wherein said administering results in no serious adverse events.

37. The method of any one of the preceding claims, wherein the Compound 1, or a pharmaceutically acceptable salt thereof, is administered without substantially inducing an acute heart rate reduction or heart block in the individual.

38. The method of any one of the preceding claims, further comprising monitoring for adverse events during the administration of the Compound 1, or a pharmaceutically acceptable salt thereof, and optionally, interrupting or terminating the administration of the Compound 1, or a pharmaceutically acceptable salt thereof.

39. The method of any one of claims 2-38, wherein the AA-associated biomarker is selected from at least one of IL-2, IL-10, IL-12, IL-13, IL-17, IL-17A, IL-22, and IFN-γ.

40. The method of any one of the preceding claims, wherein the Compound 1, or a pharmaceutically acceptable salt thereof, is the sole active ingredient in the pharmaceutical dosage form.

41. The method of any one of the preceding claims, wherein the individual has greater than or equal to 50% scalp hair loss.

42. The method of any one of the preceding claims, wherein the method is therapeutically effective to achieve at least a 50% improvement from the individual's baseline Severity of Alopecia Tool (SALT) score.

43. The method of claim 42, wherein the method is therapeutically effective to achieve at least a 50% improvement from the individual's baseline SALT score in a period of time of at least about 24 weeks.

44. The method of claim 43, wherein the method is therapeutically effective to achieve at least a 50% improvement from the individual's baseline SALT score in a period of time of about 24 weeks.

45. A compound that is (R)-2-(7-(4-cyclopentyl-3-(trifluoromethyl)benzyloxy)-1,2,3,4-tetrahydrocyclopenta[b]indol-3-yl)acetic acid (Compound 1), or a pharmaceutically acceptable salt thereof, for use in a method of treatment of alopecia areata in an individual.

46. The compound for use according to claim 45, wherein the alopecia areata is moderate to severe alopecia areata.

47. The compound for use according to claim 45 or claim 46, wherein the alopecia areata is moderate alopecia areata.

48. The compound for use according to any one of claims 45-47, wherein the use further comprises administration of a therapeutically effective amount of Compound 1 in an amount equivalent to about 0.5 to about 5.0 mg of Compound 1.

49. The compound for use according to claim 48, wherein the therapeutically effective amount is in an amount equivalent to 2 mg of Compound 1.

50. The compound for use according to claim 48, wherein the therapeutically effective amount is in an amount equivalent to 3 mg of Compound 1.

51. The compound for use according to any one of claims 45-50, wherein the Compound 1 is administered to the individual in need thereof at a frequency of once daily.

52. The compound for use according to any one of claims 45-51, wherein the Compound 1, or a pharmaceutically acceptable salt thereof, is selected from:
Compound 1;
a calcium salt of Compound 1; and
an L-arginine salt of Compound 1.

53. The compound for use according to claim 52, wherein the Compound 1, or a pharmaceutically acceptable salt thereof, is an L-arginine salt of Compound 1.

54. The compound for use according to claim 52, wherein the Compound 1, or a pharmaceutically acceptable salt thereof, is an anhydrous, non-solvated crystalline form of an L-arginine salt of Compound 1.

55. The compound for use according to claim 52, wherein the Compound 1, or a pharmaceutically acceptable salt thereof, is an anhydrous, non-solvated crystalline form of Compound 1.
